(19) United States
(12) Patent Application Publication
(10) Pub. No.: US 2009/0246788 A1
(43) Pub. Date: Oct. 1, 2009

(54) Methods and Assays for Capture of Nucleic Acids

(75) Inventors: Thomas Albert, Fitchburg, WI (US); Jigar Patel, Middleton, WI (US); Victor Lyamichev, Madison, WI (US)

Correspondence Address: BARNES & THORNBURG LLP, P.O. BOX 2786, CHICAGO, IL, 60690-2786, US

(73) Assignee: ROCHE NIMBLEGEN, INC., Madison, WI (US)

(21) Appl. No.: 12/408,485

(22) Filed: Mar. 20, 2009

Related U.S. Application Data
(60) Provisional application No. 61/041,290, filed on Apr. 1, 2008.

(51) Int. Cl. C12Q 1/68

(52) U.S. Cl. 435/6

(57) ABSTRACT

The present disclosure provides methods and systems for sequence specific nucleic acid target capture comprising enzymatic reactions. The present disclosure relates to a plurality of oligonucleotide probes for capture and subsequent detection of target nucleic acid sequences, using flap endonucleases, ligases, and/or additional enzymes, proteins or compounds, on substrates, for example microarray slides, and in solution formats.

The present application claims priority to U.S. provisional application Ser. No. 61/041,290 filed Apr. 1, 2008, which is incorporated herein by reference in its entirety.

### FIELD OF THE INVENTION

The present invention provides methods and systems for sequence specific nucleic acid target capture comprising enzymatic reactions. In particular, the present invention comprises a plurality of oligonucleotide probes for capture and subsequent detection of target nucleic acid sequences, using flap endonucleases, ligases, and/or additional enzymes, proteins or compounds on substrates, for example microarray slides, and in solution formats.

### BACKGROUND OF THE INVENTION

The advent of nucleic acid microarray technology makes it possible to build an array of millions of nucleic acid sequences in a very small area, for example on a microscope slide (e.g., U.S. Pat. Nos. 6,375,903 and 5,143,854). Initially, such arrays were created by spotting pre-synthesized DNA sequences onto slides. However, the construction of maskless array synthesizers (MAS) as described in U.S. Pat. No. 6,375,903 now allows for the in situ synthesis of oligonucleotide sequences directly on the slide itself.

Using a MAS instrument, the selection of oligonucleotide sequences to be constructed on the microarray is under software control such that it is now possible to create individually customized arrays based on the particular needs of an investigator. In general, MAS-based oligonucleotide microarray synthesis technology allows for the parallel synthesis of over 4 million unique oligonucleotide features in a very small area of a standard microscope slide. With the availability of the entire genomes of hundreds of organisms, for which a reference sequence has generally been deposited into a public database, microarrays have been used to perform sequence analysis on nucleic acids isolated from a myriad of organisms.

Nucleic acid microarray technology has been applied to many areas of research and diagnostics, such as gene expression and discovery, mutation detection, allelic and evolutionary sequence comparison, genome mapping, drug discovery, and more. Many applications require searching for genetic variants and mutations across the entire human genome; variants and mutations that, for example, may underlie human diseases. In the case of complex diseases, these searches generally result in a single nucleotide polymorphism (SNP) or set of SNPs associated with one or more diseases. Identifying such SNPs has proven to be an arduous, time consuming, and costly task wherein resequencing large regions of genomic DNA, usually greater than 100 kilobases (Kb) from affected individuals and/or tissue samples is frequently required to find a single base change or identify all sequence variants.

The genome is typically too complex to be studied as a whole, and techniques must be used to reduce the complexity of the genome. To address this problem, one solution is to reduce certain types of abundant sequences from a DNA sample, as found in U.S. Pat. No. 6,013,440. Alternatives employ methods and compositions for enriching genomic sequences as described, for example, in Albert et al. (2007, Nat. Meth., 4:903-5, Epub 2007 Oct. 14; incorporated herein by reference in its entirety) and Okou et al. (2007, Nat. Meth. 4:907-9, Epub 2007 Oct. 14; incorporated herein by reference in its entirety) and U.S. patent application Ser. Nos. 11/789,135, 11/970,949, 61/032,594 and 61/026,592; all of which are incorporated herein by reference in their entireties, disclosing alternatives that are cost-effective and rapid in effectively reducing the complexity of a genomic sample in a user defined way to allow for further processing and analysis.

However, methods in use suffer from low signal-to-noise ratios and reproducibility issues. As such, what are needed are methods and systems that allow for, for example, improvements in signal-to-noise, increase in the reproducibility from assay to assay, specific sequence capture, all while remaining quantitative. Such methods would provide maximum data utility to investigators in their endeavors to understand and identify causes of disease and associated therapeutic treatments.

### SUMMARY OF THE INVENTION

The present invention provides methods and systems for sequence specific nucleic acid target capture comprising enzymatic reactions. In particular, the present invention comprises a plurality of oligonucleotide probes for capture and subsequent detection of target nucleic acid sequences, using flap endonucleases, ligases, and/or additional enzymes, proteins or compounds on substrates, for example microarray slides, and in solution formats.

Certain illustrative embodiments of the invention are described below. The present invention is not limited to these embodiments.

In embodiments of the present invention, oligonucleotide probes are synthesized in situ, or synthesized and spotted onto a substrate, wherein said probes affixed to a substrate (e.g., microarray slide, bead, microsphere) and comprise a single stranded 5′ end complementary to target sequence, and a 3′ end comprising a hairpin structure and a terminal base complementary to a target nucleotide. In other embodiments of the present invention, probes are synthesized comprising a single stranded 5′ end further comprising a binding moiety, for example a biotin, attached to the 5′ end of the probe, and a 3′ end comprising a hairpin structure and terminal base complementary to a target sequence, and the probes are maintained in solution. In some embodiments, the hairpin structure as found in the probe comprises a sequence that is recognized and cleaved by a restriction endonuclease (RE). In some embodiments of the present invention, target nucleic acids include for example, genomic DNA or derivatives thereof, RNA, cDNA, microRNA (miRNA), noncoding RNA (ncRNA), promoter-associated small RNA (PASR), and the like are added to interact with the probes.

In some embodiments, the nucleic acids are fragmented, for example by shearing, whereas in other embodiments, nucleic acid targets are amplified, for example with PCR or by Klenow. In preferred embodiments, the target nucleic acids are labeled at the 3′ end with a detectable moiety following fragmentation or amplification, for example a fluorescent, biotin, digoxygenin, etc. moiety. The present invention is not limited by the detection moiety and/or method used, and it is contemplated that a skilled artisan will understand the myriad options of detection moieties that can be attached to the 3′ ends of nucleic acids for detection purposes. In some embodiments, the target nucleic acid sequences are at least 100 bp, at least 200 bp, at least 300 bp, at least 400 bp, at least 500 bp, at least 600 bp long. However, the present invention is not limited to the size of the target nucleic acid sequences, and non-fragmented as well as fragmented and derived nucleic acid samples (e.g., PCR amplicons, Klenow random primed amplicons, etc.) are contemplated for use with methods and assays of the present invention. In some embodiments, the target nucleic acid sequence comprises a single nucleotide polymorphism or a copy number variation.

In some embodiments, an interrogation nucleotide is positioned after the hairpin structure and at the 3′ end of the probe. In some embodiments, the interrogation nucleotide is positioned, for example, immediately prior to the hairpin structure on the 5′ side and the 3′ end of the probe is designed to be complementary to a known target sequence. In some embodiments, the proximal nucleotide is 2, 3, 4, 5, 6, 7, 8, 9, or 10 bases upstream of the double stranded hairpin structure on the 5′ side. In some embodiments, by positioning the interrogation nucleotide on the 5′ side or arm of the probe sequence, a dual specificity with regards to both the cleavase and the ligase enzymes is realized as compared to cleavase specificity alone. In some embodiments, when the interrogation nucleotide is positioned on the 5′ side as described, the cleavase specificity depends on the tripartite structures and a base specific substrate for ligation between the 3′ end of the hairpin and the 5′ end of the cleaved target.

In some embodiments, labeled target nucleic acids are incubated with the probes, either on the substrate or in solution, under conditions suitable for hybridization to occur. Following hybridization, target sequences that are complementary to the terminal 3′ nucleotide of the probe create a sequence specific structure that is recognized and cleaved by a flap endonuclease (FEN). A ligase enzyme, in preferred embodiments, a thermostable ligase enzyme, is added to the reaction for ligating the 3′ end of the probe to the 5′ end of the cleaved target nucleic acid, thereby covalently linking the target nucleic acid to the probe. If the target sequence is not complementary to the 3′ terminal nucleotide on the probe, then no cleavage structure is formed, no cleavage by the flap endonuclease occurs, and ligation cannot proceed. In some embodiments, the enzymatic reactions occur sequentially by adding the FEN to hybridized probe/target complexes, followed by separate addition of the ligase. In other embodiments, the FEN and ligase are added at the same time such that the reactions have the opportunity to occur more or less simultaneously. In some embodiments, RecJ exonuclease is added to the reaction in conjunction with a cleavase. In some embodiments, a ssDNA binding protein is additionally added to the reaction. The present invention is not limited to a particular mechanism. Indeed, an understanding of the mechanism is not necessary to practice the present invention. Nonetheless, it is contemplated that the addition of RecJ and/or a ssDNA binding protein in conjunction with a FEN provides for enhanced activity of the cleavase in that the exonuclease activity of the RecJ in degrading long overhangs of hybridized target fragments provides for substrates more suitable for optimal cleavase activity compared to reactions when RecJ and/or a ssDNA binding are absent.

In some embodiments wherein the probe is affixed to a substrate, for example a microarray slide, the non-specifically bound nucleic acid molecules are washed away from the covalently bound target molecules. In some embodiments wherein the labeled probes are maintained in solution, the labeled probes with the covalently bound target molecules are captured by a substrate, for example a bead, slide, plate, etc. coated with capture molecules. For example, if the probe is biotin labeled then beads coated with streptavidin capture the probe/target complex thereby separating the bound target molecules from the non-bound molecules. In some embodiments, the substrates are washed to further purify the captured, target molecules away from the non-specifically bound nucleic acid molecules.

In some embodiments, the substrate comprising the covalently bound targets is scanned using a fluorescent scanner, for example, to detect the fluorescent moiety found on the target sequence, and data containing sequence information is communicated to a user, for example via a computer or other visualization means.

In some embodiments, the bound target nucleic acids are released from the substrate for downstream applications such as sequencing. For example, in some embodiments the hairpin structure of the probe comprises one or more restriction endonuclease sites or uracils. The present invention is not limited to any particular cleavable sequence, and a skilled artisan will recognize the myriad of options that are amenable to methods and assays of the present invention. Once the target nucleic acid is captured, covalently bound to the probe, and separated from non-specifically bound nucleic acid molecules as described herein, the target sequence is released from the probe by digesting the probe/target complex with a RE, or uracil-DNA-glycosylase followed by endonuclease VIII digestion, wherein the sequence recognized by the RE is found in the hairpin structure of the probe or uracils were synthesized into the probe hairpin. Once released, the target sequences are eluted from the probe using methods known to those skilled in the art, for example by incubating the target sequence/probe complexes in water or a low solute solution. The eluted target molecules are applied to downstream applications, for example sequencing reactions.

Methods and assays of the present invention as described herein find utility, for example, in detecting single nucleotide polymorphisms, genomic copy number variations, and the like. Genomic anomalies can be studied for their association with diseases and disorders, thereby providing insight into the causes of diseases and disorders for research and diagnostic purposes, as well as providing potential targets for use in drug discovery in identifying therapeutic treatments for such diseases and disorders.

In some embodiments, the present invention provides a method for capturing target nucleic acid sequences comprising providing a nucleic acid sample wherein said sample comprises a detection moiety, preferably a fluorescent moiety such as Cy-3, and may or may not comprise a target sequence, at least one flap endonuclease, at least one ligase, preferably a thermostable ligase, and a plurality of oligonucleotide probes, wherein said probes comprise target sequences and a hairpin structure, applying said nucleic acid sample to said oligonucleotide probes under conditions for hybridization to occur, applying said flap and ligase enzymes to said hybridized nucleic acid/probe complex under conditions allowing for enzymatic reactions to occur thereby capturing said target nucleic acid sequences. In preferred embodiments, RecJ exonuclease and a ssDNA binding protein are included in the reaction in conjunction with a flap endonuclease. In some embodiments, the nucleic acid sample is a genomic DNA sample or a derivative thereof, wherein said sample is from a mammal, preferably a human. In some embodiments, at least one of said target sequences includes a single nucleotide polymorphism, while in other embodiments the target sequence is a genomic copy number variant. In some embodiments, the hairpin structure comprises SEQ ID NO: 1. In some embodiments, the captured target nucleic acids are further detected, for example using a fluorescent scanner. In some embodiments, the probes are affixed to a substrate, for example a microarray slide, while in other embodiments the probes are maintained in solution. In some embodiments, the probes are associated with gel pads.

In some embodiments, the present invention provides a method for capturing target nucleic acid sequences comprising providing a nucleic acid sample wherein said sample comprises a detection moiety, preferably a fluorescent moiety such as, for example, Cy-3, and may or may not comprise a target sequence, at least one flap endonuclease, at least one ligase, preferably a thermostable ligase, and a plurality of oligonucleotide probes, wherein said probes comprise target sequences and a hairpin structure wherein said hairpin structure comprises cleavable sequences, providing conditions for hybridization to occur between the probes and the target nucleic acids, applying said flap and ligase enzymes to said hybridized nucleic acid/probe complex under conditions allowing for enzymatic reactions to occur thereby capturing said target nucleic acid sequences. In preferred embodiments, RecJ exonuclease and a ssDNA binding protein are included in the reaction in conjunction with a flap endonuclease. In some embodiments, the cleavable sequences comprise a restriction endonuclease site, recognized and cleavable by a restriction endonuclease. In some embodiments, the target nucleic acids are released from the probes by restriction endonuclease digest followed by sequencing for detection of the target sequences.

In some embodiments, the present invention provides a composition for sequence specific nucleic acid capture on a substrate or in solution comprising a flap endonuclease, a ligase, and oligonucleotide probes wherein said probes comprise a hairpin structure and complementary target nucleic acid sequences. In some embodiments, the oligonucleotide probes are affixed to a substrate, for example a microarray slide or a bead.

In some embodiments, the present invention includes a kit, wherein said kit is used for capturing nucleic acid target sequences comprising at least one flap endonuclease, at least one thermostable ligase, a plurality of oligonucleotide probes affixed to a substrate or in a purified state, and at least one buffer. In preferred embodiments, RecJ exonuclease and a ssDNA binding protein are further included in a kit.

In some embodiments, the oligonucleotide probes consists essentially of a single stranded 5′ end complementary to a target sequence, and a 3′ end consisting essentially of a hairpin structure and a terminal base complementary to a target nucleotide. In some embodiments, the interrogation nucleotide is positioned on the 5′ side of the probe.

In some embodiments, the present invention includes a kit, wherein said kit is used for capturing nucleic acid target sequences and consists essentially of at least one flap endonuclease, at least one thermostable ligase, a plurality of oligonucleotide probes affixed to a substrate or in a purified state, RecJ exonuclease, a ssDNA binding protein and at least one buffer.

As used herein, the term “sample” is used in its broadest sense. In one sense, it includes a nucleic acid specimen obtained from any source. Biological nucleic acid samples may be obtained from animals (including humans) and encompass nucleic acids isolated from fluids, solids, tissues, etc. Biological nucleic acid sample may also come from non-human animals, including, but are not limited to, vertebrates such as rodents, non-human primates, ovines, bovines, ruminants, lagomorphs, porcines, caprines, equines, canines, felines, aves, etc. Biological nucleic acids may also be obtained from prokaryotes, like bacteria and other non-animal eukaryotes such as plants. It is contemplated that the present invention is not limited by the source of nucleic acids sample, and any nucleic acid from any biological Kingdom finds utility in methods as described herein.

As used herein, the term “nucleic acid molecule” refers to any nucleic acid containing molecule from any sample source, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5 -methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The used herein, the term “oligonucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. The term oligonucleotide may also be used interchangeably with the term “polynucleotide.”

As used herein, the terms “complementary” or “complementarity” are used in reference to polynucleotides related by the base-pairing rules. For example, the sequence “5′-A-G-T-3′,” is complementary to the sequence “3′-T-C-A-5′.” Complementarity may be “partial,” in which only some of the nucleic acids′ bases are matched according to the base pairing rules. Or, there may be “complete” or “total” complementarity between the nucleic acids. The degree of complementarity between nucleic acid strands has significant effects on, for example, the efficiency and strength of hybridization between nucleic acid strands, amplification specificity, etc.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. While the invention is not limited to a particular set of hybridization conditions, stringent hybridization conditions are preferably employed. Stringent hybridization conditions are sequence-dependent and will differ with varying environmental parameters (e.g., salt concentrations, and presence of organics). Generally, “stringent” conditions are selected to be about 5° C. to 20° C. lower than the thermal melting point (Tₘ) for the specific nucleic acid sequence at a defined ionic strength and pH. Preferably, stringent conditions are about 5° C. to 10° C. lower than the thermal melting point for a specific nucleic acid bound to a complementary nucleic acid. The Tₘ is the temperature (under defined ionic strength and pH) at which 50% of a nucleic acid hybridizes to a perfectly matched probe.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

By way of example, “stringent conditions” or “high stringency conditions,” comprise hybridization in 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 mg/ml), 0.1% SDS, and 10% dextran sulfate at 42° C., with washes at 42° C. in 0.2×SSC (sodium chloride/sodium citrate) and 50% formamide at 55° C., followed by a wash with 0.1×SSC containing EDTA at 55° C. For moderately stringent conditions, it is contemplated that buffers containing 35% formamide, 5×SSC, and 0.1% (w/v) sodium dodecyl sulfate are suitable for hybridizing at 45° C. for 16-72 hours. Furthermore, it is contemplated that formamide concentration may be suitably adjusted between a range of 0-45% depending on the probe length and the level of stringency desired. In some embodiments of the present invention, probe optimization is obtained for longer probes (for example, greater than 50 mers) by increasing the hybridization temperature or the formamide concentration to compensate for a change in the probe length. Additional examples of hybridization conditions are provided in many reference manuals, for example in “Molecular Cloning: A Laboratory Manual”, as referenced and incorporated herein.

Similarly, “stringent” wash conditions are ordinarily determined empirically for hybridization of target sequences to a corresponding probe array. For example, the arrays are first hybridized and then washed with wash buffers containing successively lower concentrations of salts, or higher concentrations of detergents, or at increasing temperatures until the signal-to-noise ratio for specific to non-specific hybridization is high enough to facilitate detection of specific hybridization. By way of example, stringent temperature conditions will usually include temperatures in excess of about 30° C., more usually in excess of about 37° C., and occasionally in excess of about 45° C. Stringent salt conditions will ordinarily be less than about 1000 mM, usually less than about 500 mM, more usually less than about 150 mM. Stringent wash and hybridization conditions are known to those skilled in the art, and can be found in, for example, Wetmur et al., 1966, J Mol Biol 31:349-70 and Wetmur, 1991, Crit Rev Bio Mol Biol 26:227-59; incorporated herein by reference in their entireties.

It is well known in the art that numerous equivalent conditions may be employed to adjust and regulate stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered. As such, the components and concentrations of hybridization and wash solutions will vary to generate conditions of stringency. In preferred embodiments of the present invention, hybridization and wash solutions are utilized as found commercially available through Roche-NimbleGen (e.g., NimbleChip™ CGH Arrays, NimbleGen Hybridization Kits, etc.).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded, however in the present invention the probes are intended to be single stranded. Probes are useful in the detection, identification and isolation of particular gene sequences. Probes, in embodiments of the present invention, comprise a 5′ single stranded end and a 3′ end comprising a hairpin structure. A restriction endonuclease site may or may not be present in the hairpin structure.

As used herein, the term “derivative thereof” of “portion” or “fragment” when in reference to a nucleotide sequence (as in “a derivative thereof of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.). Fragments can be obtained through, for example, sonication, PCR amplification, Klenow amplification, or any other means known in the art for reducing a nucleotide sequence to smaller sequences thereof. In the present invention, fragments, derivatives of nucleic acid sequences are preferably at least 200 bp, at least 300 bp, at least 400 bp, at least 500 bp, at least 600 bp. However, the present invention is not limited to the size of the target nucleic acid sequences.

As used herein, the term “purified” or “to purify” refers to the removal of components (e.g., contaminants) and/or contaminants from a sample. The term “purified” refers to molecules, either nucleic or amino acid sequences that are removed from their natural environment, isolated or separated. An “isolated nucleic acid sequence or sample” is therefore a purified nucleic acid sequence or sample. “Substantially purified” molecules are at least 60% free, preferably at least 75% free, and more preferably at least 90% free from other components with which they are naturally associated. In certain embodiments of the present invention, “purified” relates to the separation of unbound sample nucleic acid molecules and reaction components (e.g., enzymes, etc.) away from probe/target complexes, typically by washing with wash buffers of one or more stringencies, thereby “purifying” the probe/target nucleic acid complexes from other reaction components.

As used herein, the term “interrogation nucleotide” refers to the nucleotide in the probe that interacts with (e.g, matching base pair formation or a mismatch) the target sequence to determine a specific mutation such as a single nucleotide polymorphism or for sequence-specific capture of target nucleotides from a sample using a plurality of oligonucleotide probes. In some embodiments, the interrogation nucleotide is positioned as a terminal base at the 3′ end of the probe. In some embodiments, the interrogation nucleotide is positioned on the 5′-side or arm of the probe (i.e., the single stranded region of the probe), proximal to the hairpin stem structure. In some embodiments, interrogation nucleotide is positioned immediately next to and upstream of the hairpin stem structure. In some embodiments, the proximal nucleotide is 2, 3, 4, 5, 6, 7, 8, 9, or 10 bases upstream of the double stranded hairpin structure on the 5′ side. The interrogation nucleotide may also be referred as “allele specific nucleotide”. In some embodiments, the interrogation nucleotide, if a corresponding complementary nucleotide is present in the target, creates an overlapping tripartite structure with the target molecule that is recognized by a cleavase.

### DESCRIPTION OF THE FIGURES

FIG. 1 shows an exemplary embodiment for the use of flap endonucleases and ligases to cleave and ligate target molecules to probes affixed to a microarray solid support; A) a four probe set for one strand of a target DNA molecule associated with potential target DNA sequences, B) flap endonuclease cleavage of the correct target nucleic acid sequence associated with the complementary probe, with no cleavage of the incorrect target sequence, and C) ligation of the 5′ end of the correct target sequence with the 3′ end of the probe, with the incorrect target sequences not ligated to the probe.

FIG. 2 demonstrates an exemplary embodiment for the use of flap endonucleases and ligases to cleave and ligate target molecules to labeled probes in solution; A) sequence specific cleavage followed by ligation of target molecules (SEQ ID NO: 33) to a biotinylated probe (SEQ ID NO: 32) when an invasive complex is formed, and B) capture of labeled probes with covalently attached target molecules to beads coated with streptavidin.

FIG. 3 shows an exemplary hairpin sequence of an oligonucleotide probe (SEQ ID NO: 1) as described herein.

FIG. 4 shows experimental data of using the present method in capturing CPK6 target sequences; target sequences are captured by probes with hairpins, FEN cleaved, and ligated to the probe, whereas control sequences are not.

FIG. 5 shows experimental data demonstrating the efficiency of methods of the present invention in capturing target sequences (correct vs. incorrect base call), and the fold difference in capturing the correct versus the incorrect target sequence.

FIG. 6 demonstrates the three different E-coli amplifications and the restriction digest maps for created fragmented experimental DNAs.

FIG. 7 exemplifies the use of the present invention is identifying genomic mutations.

FIG. 8 demonstrates the effect of 5′ flap length on the activity of different cleavase molecules with regards to the ability to correctly perform base calling as indicated by low discrimination scores (<0.5 D score).

FIG. 9 exemplifies the effect of RecJ in enhancing cleavase activity; A) demonstrates cleavase reactions without RecJ and B) demonstrates the enhanced activity of a cleavase in the presence of RecJ.

FIG. 10 illustrates a comparison of hairpin configuration for single vs. dual enzymatic specificity in cleavase and ligase reactions. A, C) Hairpin configurations have, for example, oligomer probes (SEQ ID NOS: 11-14, 34-37) synthesized from 5′-3′ with a hairpin of stem-length (6 bp-12 bp) with a 1 bp overhang on the 3′ end. B, D) Hairpin configurations have, for example, oligomer probes (SEQ ID NOS: 16-19, 38-41) synthesized from 5′-3′ with a hairpin of stem-length (6 bp-12 bp). These generate base specific substrates for ligation between the 3′ end of the hairpin and the 5′ end of the cleaved targets (SEQ ID NOS: 15, 42). OL refers to overlap at an interrogation site. Therefore, OL1 refers to an overlap of 1 nucleotide and OL0 refers to zero or no overlap at the interrogation site.

FIG. 11 shows the evaluation of specificity of base calling using various hairpin configurations across an 83 bp PCR fragment. Discrimination scores were calculated for every base across the 83 bp fragment and plotted against two classes of hairpin configurations; 1) Dual (OL0) and 2) Single (OL1) enzyme specificity. Within each class, several stem and loop sequences were compared. The control probeset with no hairpins (No_HP) had the lowest discrimination, whereas the dual enzymatic hairpin configuration had the highest.

FIG. 12 shows the evaluation of specificity of base calling using various hairpin configurations across an 83 bp PCR fragment. Discrimination scores were calculated for every base across the 83 bp fragment and plotted against two classes of hairpin configurations; 1) Dual (OL0) (SEQ ID NOS: 20-25) and 2) Single (OL1) (SEQ ID NOS: 26-31) enzyme specificity. Within each class, several stem and loop sequences were compared.

### DETAILED DESCRIPTION OF THE INVENTION

The present invention provides methods and assays for the use of flap endonucleases and ligases in methods and assays for targeted capture of nucleic acids on solid substrates and in solution. Certain illustrative embodiments of the invention are described below. The present invention is not limited to these embodiments.

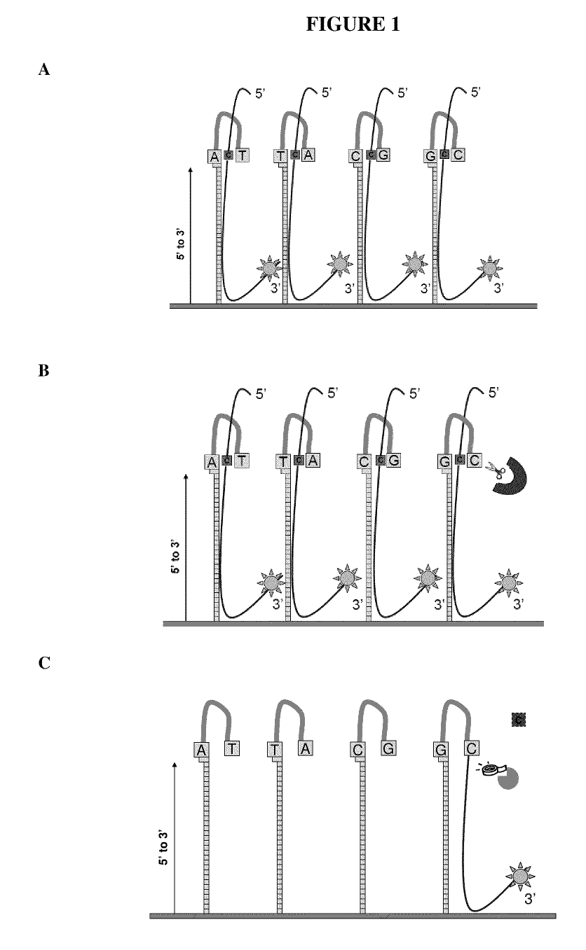

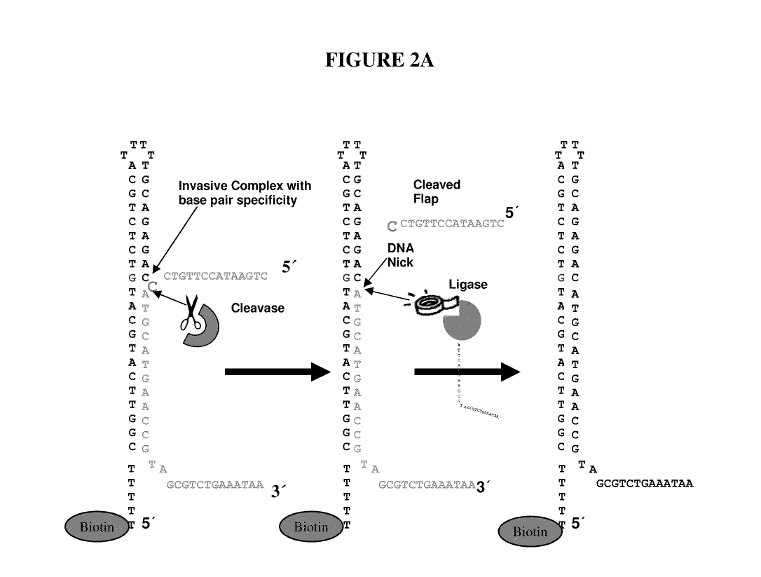

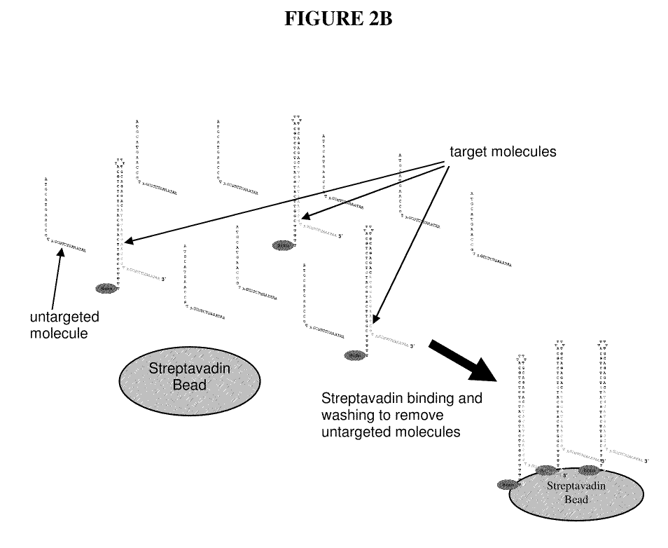

Flap endonucleases (FENs), also known as cleavases, are structure specific enzymes that are capable of cleaving nucleic acids in a sequence specific manner. The enzymes have been used as components in the development of the Invader® genotyping and nucleic acid detection technology from Third Wave Technologies, for example as found in US patents and Published Patent Applications U.S. Pat. Nos. 5,843,669, 5,888,780, 6,090,606, 6,562,611, 7,122,364, 2007/0003942, 2007/0292856, 2006/0292580, 2006/0183207, 2006/0177835, 2006/0154269 and 2006/0040294, all of which are incorporated herein by reference in their entireties. Additional examples of flap endonuclease compositions and methods are found in US patents and Published Patent Applications U.S. Pat. Nos. 6,255,081, 6,251,649, 6,979,725, 5,874,283, 2007/0292934, 2007/0292864, 2007/0231815, and 2007/0105138, all of which are incorporated herein by reference in their entireties. The endonuclease activity of FENs includes recognition of a DNA duplex which has a 5′ overhang (flap) on one of the strands, called the invasive complex (as exemplified in FIGS. 1 and 2). The FEN catalyses hydrolytic cleavage of the phosphodiester bond at the junction of the single and double stranded DNA (Harrington and Lieber, 1994, EMBO J, 13:1235-46; Harrington and Lieber, 1995, J Biol Chem 270:4503-8; incorporated herein be reference in their entireties). The ability of FENs to recognize and cleave specific secondary structures allows these enzymes to be used to detect internal sequence differences in nucleic acids without prior knowledge of the specific sequence of the nucleic acid. In some embodiments of the present invention, when the 3′ terminal nucleotide of the probe is present in the target nucleic acid sample, the specific structure recognized by FENs is created and FEN cleavage occurs. However, when the sequence as found in the probe is not complementary to the target sequence, no structure is realized and FEN cleavage does not occur. As such, sequence specific recognition of target nucleic acids is achieved.

Ligases, in particular thermostable ligases, are further contemplated for use with methods and assays of the present invention. Ligation of the probe 3′ end to the 5′ end of the target molecule is performed following FEN cleavage of the target molecule, if the FEN recognized structure is present. As known to a skilled artisan, ligases catalyse the formation of covalent phosphodiester bonds between juxtaposed 3′ hydroxyl and 5′ phosphate termini in duplex DNA or RNA (exemplified in FIGS. 1 and 2). In preferred embodiments of the present invention, thermostable ligases are contemplated. Non-thermostable ligases, such as T4 DNA ligase demonstrate optimal enzymatic activity at room temperature. Thermostable ligases, for example those found in the bacteria Thermus aquaticus and Pyrococcus furiosus, demonstrate optimal enzymatic activity at much higher temperatures, for example greater than 45° C., allowing more flexibility in temperature conditions when applied to methods and assays of the present invention as described herein. Examples of thermostable ligases are found in, for example, US patents and Published Patent Applications U.S. Pat. Nos. 6,949,370, 6,576,453, 6,280,998, 6,444,429, 5,700,672, 2007/0037190, 2005/0266487 and European Patent Publication WO07/035439, all of which are incorporated herein by reference in their entireties. Once FEN has cleaved that target nucleic acid, ligation via a ligase enzyme covalently links the target sequence to the probe, wherein said probe is either affixed to a solid support, for example a microarray slide, or kept in solution. As such, sequence specific capture of a target nucleic acid is achieved.

It was further contemplated that the inclusion of RecJ with or without a ssDNA binding protein (SSBP) would enhance the efficacy of the cleavase once the invasive complex in the described hybridization reaction is recognized by the cleavase. Rec J exonuclease (RecJ) degrades single stranded DNA in the 5′-3′ direction and further participates in mismatch repair and homologous recombination (Lovett et al., 1989, Proc Natl Acad Sci 86:2627-2631; Yamagata et al., 2001, Nucl Acids Res 29:4617-4624; Han et al., 2006, Nucl Acids Res 34:1084-1091; incorporated herein by reference in their entireties). Rec J degrades both phosphorylated and unphosphorylated DNA ends with equal affinity, however requires that the single stranded end be at least 7 nucleotides long. Rec J is a processive exonuclease and degrades approximately 1000 nucleotides after it binds to the single stranded end, typically stopping when it comes to the double stranded DNA junction. However, RecJ nuclease activity is not precise, and it may stop degradation activity a few nucleotides short of the junction or a few base pairs after the function. It has been suggested that the recruitment of Rec J to the single stranded DNA may be enhanced by the addition of a single stranded DNA binding protein to the degradation reaction (Han et al, 2006). As such, the inclusion of one or more of a RecJ exonuclease and a SSBP is contemplated to enhance the cleavase activity allowing for higher efficiencies is sequence specific capture of a target nucleic acid as described herein.

In some embodiments, oligonucleotide probes are attached to a solid support, such as a microarray slide, at their 5′ ends or 5′ sides or 5′ arms, wherein said probes comprise a central portion that is single stranded and complementary to a target sequence, and a 3′ terminal region that comprises a hairpin structure comprising a loop of single nucleotides bounded by double stranded region of complementary nucleic acids, ending in a 3′ terminal base specific to a target sequence of interest (e.g., an interrogation nucleotide for determining a genomic mutation such as single nucleotide polymorphism). In some embodiments, the base that is specific to the target sequence, e.g., for determining a mutation or a polymorphism is placed immediately proximate to the hairpin loop structure on the 5′-side of the probe. In preferred embodiments, the double stranded hairpin region is at least 3 base pairs, at least 5 base pairs, at least 6 base pairs, at least 8 base pairs, at least 10 base pairs, at least 16 base pairs. The nucleic acid probes are incubated with a complex target population of nucleic acid strands (e.g., DNA, cDNA, gDNA, RNA, mRNA, tRNA, etc.), for example human genome DNA (gDNA) (e.g., whole or fragmented), under conditions that favor hybridization of complementary DNA strands such that nucleic acid sequences complementary to the single stranded portion of the 5′ end of the nucleic acid probe and the probe 3′ terminal base specifically hybridize to the probe. In some embodiments, the target nucleic acids are labeled on the 3′ end with, for example, a detectable moiety (e.g., fluorophore, chromophore, radioisotope, etc.).

In some embodiments, the present invention provides oligonucleotide probes attached to a solid support, such as a microarray slide, at their 5′ ends, wherein said probes comprise a central portion that is single stranded and partially complementary to a target sequence, and a 3′ terminal region that comprises a hairpin structure comprising a loop of single nucleotides bounded by a double stranded region of complementary nucleic acids, ending in a 3′ terminal base complementary to a target sequence, wherein a nucleotide proximal to the 5′ end of the double stranded region of complementary nucleic acids that comprise a hairpin structure includes a sequence complementary to a target sequence of interest (e.g., an interrogation nucleotide for determining genomic mutations such as single nucleotide polymorphisms). In preferred embodiments, the proximal nucleotide is immediately proximate to the double stranded hairpin structure. In some embodiments, the proximal nucleotide is 1, 2, 3, 4, 5, 6, 7, 8, 9, or 10 bases upstream of the double stranded hairpin structure on the 5′ side. In preferred embodiments, the double stranded hairpin region is at least 3 base pairs, at least 10 base pairs, at least 16 base pairs. The nucleic acid probes are incubated with a complex target population of nucleic acid strands (e.g., DNA, cDNA, gDNA, RNA, mRNA, tRNA, etc.), for example human genome DNA (gDNA) (e.g., whole or fragmented), under conditions that favor hybridization of complementary DNA strands such that nucleic acid sequences complementary to the single stranded portion of the 5′ end of the nucleic acid probe and the probe 3′ terminal base specifically hybridize to the probe. In some embodiments, the target nucleic acids are labeled on the 3′ end with, for example, a detectable moiety (e.g., fluorophore, chromophore, radioisotope, etc.).

In some embodiments, the present invention provides oligonucleotide probes attached to a solid support, such as a microarray slide, at their 5′ ends, wherein said probes comprise a central portion that is single stranded and complementary to a target sequence, and a 3′ terminal region that comprises a hairpin structure comprising a loop of single nucleotides bounded by a double stranded region of complementary nucleic acids, ending in a 3′ terminal base complementary to a target sequence. In preferred embodiments, the double stranded hairpin region is at least 3 base pairs, at least 10 base pairs, at least 16 base pairs. The nucleic acid probes are incubated with a complex target population of nucleic acid strands (e.g., DNA, cDNA, gDNA, RNA, mRNA, tRNA, etc.), for example human genome DNA (gDNA) (e.g., whole or fragmented), under conditions that favor hybridization of complementary DNA strands such that nucleic acid sequences complementary to the single stranded portion of the 5′ end of the nucleic acid probe and the probe 3′ terminal base specifically hybridize to the probe. In some embodiments, the target nucleic acids are labeled on the 3′ end with, for example, a detectable moiety (e.g., fluorophore, chromophore, radioisotope, etc.). In some embodiments, oligonucleotide probes comprising a central portion that is single stranded and complementary to a target sequence, and a 3′ terminal region that comprises a hairpin structure comprising a loop of single nucleotides bounded by a double stranded region of complementary nucleic acids, ending in a 3′ terminal base complementary to a target sequence find utility in hybridization assays for determining genetic anomalies such as deletions, translocations, and the like.

In some embodiments, oligonucleotide probes are synthesized and affixed to a substrate, for example a microarray slide or chip, as described in DNA Microarrays: A Molecular Cloning Manual, 2003, Eds. Bowtell and Sambrook, Cold Spring Harbor Laboratory Press, incorporated herein by reference in its entirety. In preferred embodiments, capture oligonucleotide probes are synthesized directly on a substrate, such as a microarray slide, using maskless array synthesizers (MAS), for example as described in US patents and Patent Publications U.S. Pat. Nos. 7,157,229, 7,083,975, 6,444,175, 6,375,903, 6,315,958, 6,295,153, 5,143,854, 2007/0037274, 2007/0140906, 2004/0126757, 2004/0110212, 2004/0110211, 2003/0143550, 2003/0003032, and 2002/0041420, all of which are incorporated herein by reference in their entireties. When using a MAS instrument to print microarrays, the selection of oligonucleotide probe sequences are constructed in situ directly on the microarray slide under software control, such that individually customized arrays based on the particular needs of an investigator are created. Such arrays comprise hundreds, thousands, and millions of probes.

Oligonucleotide probes are typically synthesized from 3′ to 5′, whether synthesized in situ (e.g., MAS synthesis) on a substrate (e.g., microarray slide) or synthesized and then spotted onto a substrate. However, as most nucleic acid enzymes (e.g., restriction endonucleases, polymerases, terminal transferase, ligases, kinases, phosphatases, etc.) have activities from 5′ to 3′, enzymatic reactions require synthesis of probes using reverse chemistries (e.g., from 5′ to 3′). As such, method and assay embodiments of the present invention comprise in situ synthesis of oligonucleotide probes synthesized preferentially from 5′ to 3′ using MAS instruments as described in, for example, Albert et al. (2003, Nucl Acids Res 31: e35; incorporated herein by reference in its entirety).

The present invention provides methods and assays for detecting differences in nucleic acid sequences, for example single nucleotide polymorphisms, genomic copy number variants, methylation status, etc. In methods and assays of the present invention, target nucleic acids are hybridized with probes, wherein said probes are affixed (e.g., via in situ synthesis or otherwise) to a substrate or found in solution. In some embodiments, the probes are at least 15 nucleotides (nts) long, at least 20 nts, at least 25 nts, at least 30 nts, at least 35 nts, at least 40 nts, at least 45 nts, at least 50 nts, at least 55 nts, at least 60 nucleotides long. In some embodiments, probes of the present invention are synthesized in situ on a substrate, for example a microarray slide, microarray chip, bead, plate, etc. In preferred embodiments, the probes are synthesized in situ using MAS instrumentation wherein the 5′ terminus of the probe is affixed to the substrate. In some embodiments, the probes are synthesized in solution and maintained in solution.

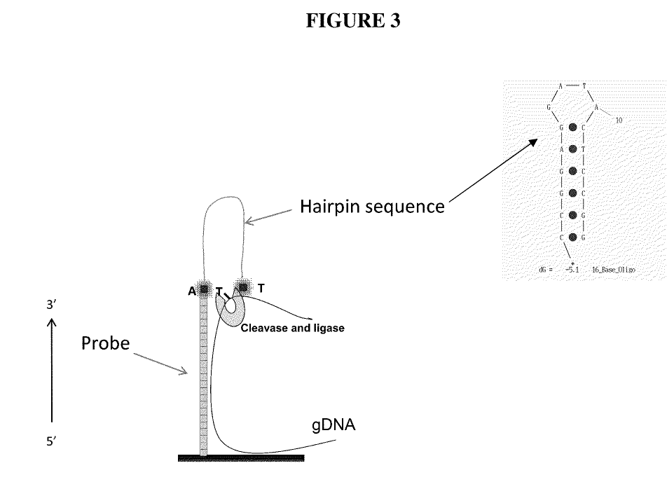

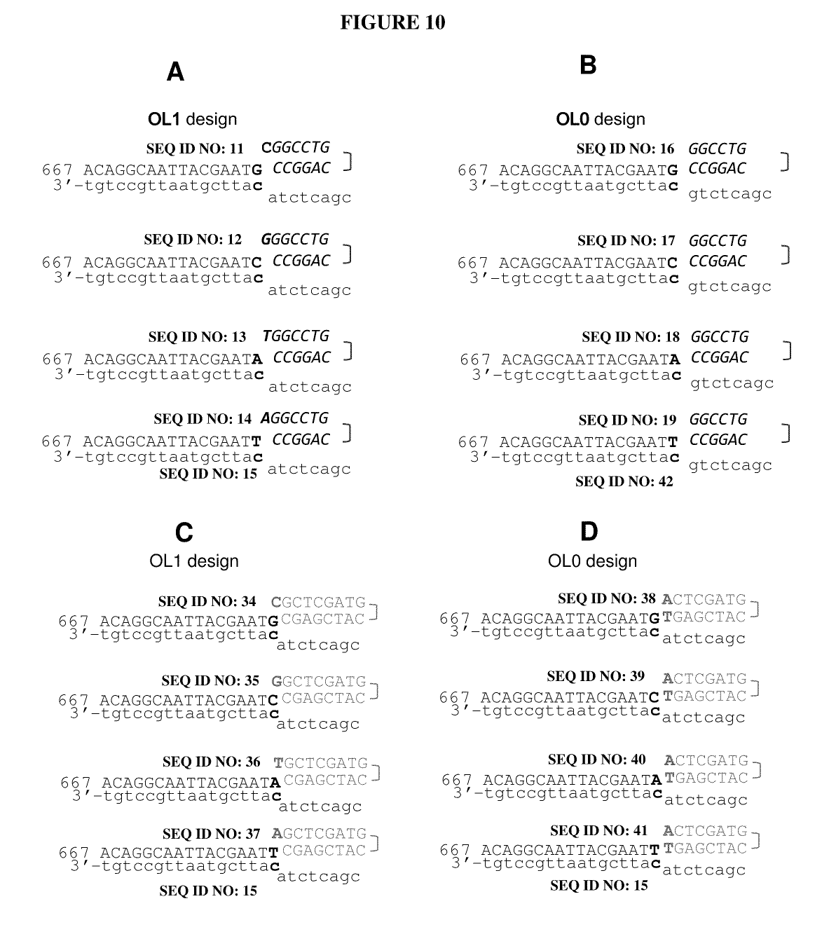

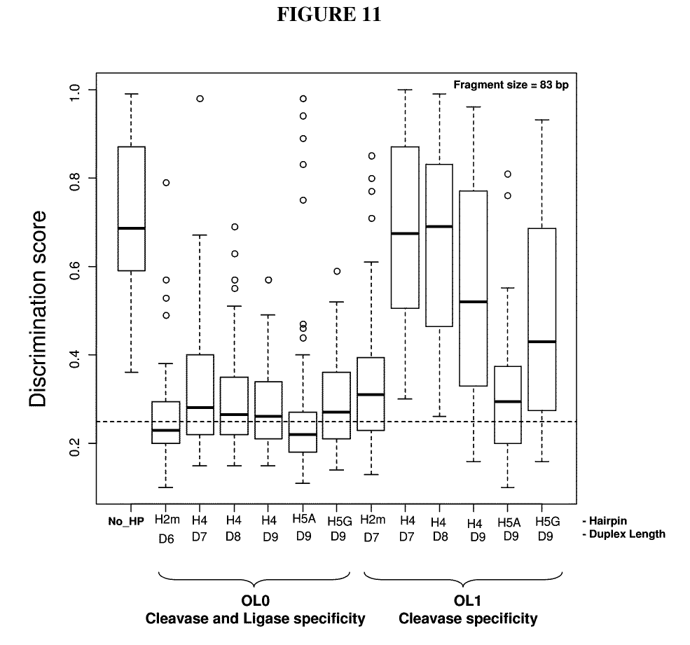

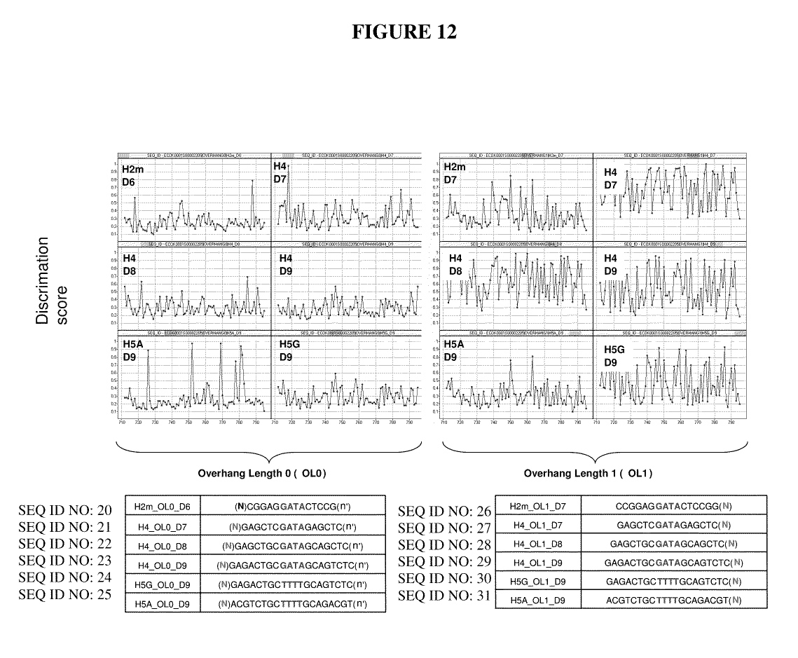

Probes of the present invention comprise a single stranded 5′ end complementary to a target sequence, a 3′ end that comprises a hairpin structure comprising a series of complementary bases that create a double stranded region and sequences that are not complementary that create a single stranded region amid the double stranded region (e.g., hairpin structure), and a 3′ terminal base complementary to a specific target sequence. In some embodiments, the hairpin structure is at least 5 bases, 10 bases, at least 12 bases, at least 14 bases, at least 16 bases, at least 18 bases long. In some embodiments, the hairpin structure is preferably 16 bases. In some embodiments, the hairpin structure comprises SEQ ID NO: 1, as exemplified in FIG. 3. However, the present invention is not limited by the sequence of the hairpin structure, and further examples of hairpin structures and their design are found in Varani (1995, Ann Rev Biophys Biomol Struct 24:379-404) and Antao et al. (1991, Nucl Acids Res 19:5901-5), both of which are incorporated herein by reference in their entireties. As exemplified in FIGS. 1 and 2, the nucleotide base on the probe that is complementary to the 3′ terminal base of the probe hairpin determines assay specificity, and is sometimes referred to as the “allele specific base”. As exemplified in FIG. 10, the nucleotide base on the 5′-side or arm of the probe proximal to the hairpin structure also determines assay specificity. When the allele specific base is complementary to the corresponding base of the target strand, the hairpin and target molecules form a specific structure, termed the “invasive complex” that is recognized by FEN enzymes. When the base specific nucleotide is present in the target strand FEN cleaves the target strand on the 3′ side of the base thereby releasing the non-hybridized, or flap, 5′ end of the target strand. If the base is not complementary to the allele specific base, then the invasive complex does not form, and the target molecule is not cleaved by the FEN.

In some embodiments, after hybridization of the probe with the target nucleic acid, the probe/target complex is incubated with a cocktail comprising one or more of a flap endonuclease (FEN), ligase, Rec J, a ssDNA binding protein, and appropriate buffers and cofactors (ATP or NAD+) necessary for enzymatic reactions to occur. The FEN cleaves target molecules that form the invasive complex, resulting in a gap between the nucleic acid probe hairpin and the target nucleic acid molecule. It is contemplated that the inclusion of RecJ with or without a ssDNA binding protein increases the efficiency of the cleavase reaction thereby allowing for increased sensitivity in the cleaving of the invasive complexes. Ligase repairs the gap, covalently attaching the target molecule to the probe. If the invasive complex does not form, then the FEN does not cleave and the target molecule is not ligated to the probe. Unbound molecules are subsequently separated from the bound targets by washing. Since the target molecules are covalently attached to the probes on the substrate, washing can be performed with much higher stringency that those afforded to non-covalently bound hybridization methods, thereby increasing the removal of non-specifically bound, non-target molecules and greatly improving capture specificity and signal-to-noise ratios.

In some embodiments, target molecules that bind to the probe are identified by a detectable means (e.g. colorimetry, radiometry, fluorometry, gel electrophoresis, etc.) using data analysis instruments and software as described herein and as known to a skilled artisan.

As the target molecules are covalently bound to the probes, the arrays are capable of being washed with high stringency for removal of unbound, non-target molecules thereby allowing for increased reduction of background signal and increasing an assay's signal-to noise ratio. Applications of the present invention include, but are not limited to, comparative genomic hybridization (CGH), single nucleotide polymorphism genotyping, gene transcription profiling, genome methylation analysis, chromatin immunoprecipitation mapping, and the like.

In some embodiments, the present invention provides assays and methods for target capture of DNA for, for example, high throughput sequencing applications and other downstream applications.

In some embodiments, nucleic acid probes are in solution and bound at their 5′ ends to a capturable moiety, for example a biotin moiety (FIG. 2). The nucleic acid probes further comprise a 5′ region that is single stranded and complementary to a target sequence and a 3′ terminal region that comprises a hairpin structure comprising a loop of single nucleotides bounded by a double stranded region of complementary nucleic acids as previously described. In some embodiments, the hairpin structure is at least 5 bases, 10 bases, at least 12 bases, at least 14 bases, at least 16 bases, at least 18 bases long. In some embodiments, the probes are at least 15 nucleotides (nts) long, at least 20 nts, at least 25 nts, at least 30 nts, at least 35 nts, at least 40 nts, at least 45 nts, at least 50 nts, at least 55 nts, at least 60 nucleotides long. It is contemplated that the probe is preferably less than 100 bases. In some embodiments, the hairpin sequences comprise cleavable sequences for releasing the bound target sequences from the probe following ligation. For example, the hairpin sequences comprise a restriction endonuclease (RE) site or one or more uracils. The nucleic acid probes are incubated with a complex target population of nucleic acid strands (e.g., DNA, cDNA, gDNA, RNA, mRNA, tRNA, etc.), for example fragmented human genome DNA (gDNA), under conditions that favor hybridization of complementary DNA strands such that fragments of the genome complementary to the single stranded portion of the 5′ end of the nucleic acid probe specifically hybridize to the probe. In some embodiments, the target nucleic acids are labeled on the 3′ end with, for example, a detectable moiety (e.g., fluorophore, chromophore, radioisotope, etc.).

When the allele specific base is complementary to the corresponding base of the target strand, as seen in FIG. 2, the hairpin and target molecules hybridize and form a specific structure that is recognized by FEN enzymes. When the base specific nucleotide is present in the target strand (FIG. 2) FEN cleaves the target strand on the 3′ side of the base thereby releasing the non-hybridized, or flap, 5′ end of the target strand. If the base is not complementary to the allele specific base, then the invasive complex does not form, and the target molecule is not cleaved by the FEN. It is contemplated that the incorporation of RecJ with or without a ssDNA binding protein in conjunction with a FEN will increase the efficiency of the cleavase enzyme.

In some embodiments, following hybridization, the probe complex is incubated with a cocktail comprising one or more of a FEN enzyme, ligase enzyme, RecJ, a ssDNA binding protein, appropriate buffers and cofactors (ATP or NAD+) necessary for the required enzymatic reactions to occur. The FEN cleaves target molecules that form the invasive complex, resulting in a gap between the nucleic acid probe hairpin and the target nucleic acid molecule. As previously described, the ligase repairs the gap by covalently attaching the target molecule to the probe. If the invasive complex does not form, then the FEN does not cleave and the target molecule is not ligated to the probe. In some embodiments, as exemplified in FIG. 2B, the target molecules that are covalently attached to a labeled probe in solution, in this instance a biotin labeled probe, are captured (e.g., bound) by streptavidin (SA) coated beads, wherein streptavidin binds the biotin of the labeled probe as known to those skilled in the art. Following streptavidin binding, the beads are removed from solution and washed thereby removing unbound, untargeted molecules from the bound target molecules on the captured beads and purifying the target molecule/probe complexes away from unwanted reaction components. Since the target molecules are covalently attached to the SA bound biotin labeled probe, washing can be performed with much higher stringency that those afforded to non-covalently bound hybridization methods, thereby increasing the removal of non-specifically bound, non-target molecules and greatly improving capture specificity and signal-to-noise ratios.

In some embodiments, the target molecules are released from the SA bound probe by exposing the beads comprising the target molecules to a restriction endonuclease, for example when the recognition site of the RE is incorporated into the hairpin sequence of the probe during synthesis. In some embodiments, if one or more uracils are incorporated into the hairpin during synthesis, the target molecules are released from the SA bound probes by incubation with uracil-DNA-glycosylase (UDG) followed by Endonuclease VIII digestion at the damaged site. The present invention is not limited to the method of release of the target molecule from the bound probe and other methods know in the art to cleave DNA are contemplated for use with methods and assays of the present invention.

Once liberated, the targeted molecules are applied to downstream applications, such as sequencing using, for example, a high-throughput sequencer. Methods and assays of the present invention further provide an investigator the ability to precisely define the end point and directionality of each captured target nucleic acid molecule and, as a consequence, each sequencing read. It is contemplated that release of the target sequence using enzymatic means as described herein is amenable to release of target sequences from probes wherein said probes are synthesized on a solid support, such as a microarray slide, as described herein.

In some embodiments, methods and assays of the present invention provide for analysis of single nucleotide polymorphisms (SNPs). In some embodiments, the present invention provides methods and assays for analysis of copy number variation (CNV) in DNA samples, for example genomic DNA samples. SNP and CNV analysis is useful in association studies, for example between different species and between SNPs and CNVs associated with diseases and disorders. As such, methods and assays of the present invention provide for the analysis of genomic variation and their association with diseases in a particular subject, for example a human subject. However, it is contemplated that the present invention is not limited to analysis of genomic sequences from any particular genus and/or species, for example any prokaryotic or eukaryotic sequence is considered amenable to applications of the present invention, for research, diagnostic or therapeutic use.

In some embodiments, the present invention is not limited to a particular set of hybridization conditions. However, stringent hybridization conditions as known to those skilled in the art are preferably employed. Hybridization solutions of use with the present invention include, but are not limited to, those found in NimbleGen Hybridization Kits (Roche NimbleGen, Madison Wis.). In some embodiments, the present invention provides washing the probe/target complexes following enzymatic cleavage and ligation reactions thereby removing unbound and non-specifically bound nucleic acid molecules. In some embodiments, the present invention provides washes of differential stringency, for example a wash buffer I comprising 0.2×SSC, 0.2% (v/v) SDS, and 0.1 mM DTT, a wash buffer II comprising 0.2×SSC and 0.1 mM DTT and a wash buffer III comprising 0.5×SSC and 0.1 mM DTT. In some embodiments, solutions and buffers for washing, hybridization, and enzymatic reactions comprise lithium. The present invention is not limited by composition of the hybridization and/or wash buffers. In some embodiments, the target sequences are eluted from the probes following, for example RE digestion, using, for example, water or similar low solute solutions known to those skilled in the art.

In some embodiments, the present invention provides capture of target nucleic acid sequences for subsequent use in targeted array-based-, shotgun-, capillary-, or other sequencing methods known to the art. As known to a skilled artisan, sequencing by synthesis is understood to be a sequencing method which monitors the generation of side products upon incorporation of a specific deoxynucleoside-triphosphate during the sequencing reaction (Rhonaghi et al., 1998, Science 281:363-65; incorporated herein by reference in its entirety). For example, one or the more prominent embodiments of the sequencing by synthesis reaction is the pyrophosphate sequencing method. In pyrosequencing, generation of pyrophosphate during nucleotide incorporation is monitored by an enzymatic cascade which results in the generation of a chemo-luminescent signal. The 454 Genome Sequencer System (Roche Applied Science cat. No. 04760085001) is based on the pyrophosphate sequencing technology. For sequencing on a 454 GS20 or 454 FLX instrument, the average genomic DNA fragment size is preferably in the range of 200 or 600 bp, respectively. Sequencing by synthesis reactions can also comprise a terminator dye type sequencing reaction. In this case, the incorporated dNTP building blocks comprise a detectable label, such as a fluorescent label, that prevents further extension of the nascent DNA strand. The label is removed and detected upon incorporation of the dNTP building block into the template/primer extension hybrid, for example, by using a DNA polymerase comprising a 3′ -5′ exonuclease or proofreading activity. However, the present invention is not limited by the type of downstream application that may used in conjunction with the present invention.

In some embodiments, the target sequences are released from the oligonucleotide probe by enzymatic digest (e.g., restriction endonuclease, UDG and Endo VIII, etc.) and eluted away from the probe and sequenced. In some embodiments, the sequencing is performed using a 454 Life Sciences Corporation sequencer. In some embodiments, the present invention provides target sequence amplification following elution by emulsion PCR (emPCR) following manufacturer's protocols. The beads comprising the clonally amplified target nucleic acids from the emPCR are transferred into a picotiter plate according to the manufacturer's protocol and subjected to a pyrophosphate sequencing reaction for sequence determination.

In some embodiments, the present invention provides methods and assays wherein a plurality of different target sequences is contemplated for detection on one array or in one solution, for example for concurrent capture and detection of multiple target sequences. In such embodiments, a two color labeling is contemplated (e.g., two channel fluorescence, fluorescent/non-fluorescent, etc.). For example, one target sequence is labeled with one detectable moiety and another target sequence is labeled with a second detectable moiety. For example, one target sequence is labeled with a fluorescent moiety (e.g., fluorescein, Cy-3, Cy-5, etc.) and the second target sequence is labeled with a non-fluorescent moiety (e.g., biotin, digoxygenin) or a fluorescent moiety differing in wavelength detection from the first fluorescent moiety. Terminal transferase can be used to 3′ end label the target sequences by using, for example, ddCTP conjugates of the fluorophore (e.g., fluorescein-12-ddCTP) and the second moiety (e.g., biotin-11-ddCTP). As such, dual channel detection or differential moiety detection allows for differentiation between two different captured target sequences.

In some embodiments, the signal detected upon completion when practicing methods and assays of the present invention as described herein are further amplified. Examples of signal amplification methods include, but are not limited to, those found in Tyramide Signal Amplification kits commercially available through, for example NEN® Life Sciences Products, Inc. (Boston Mass.).

In some embodiments, data analysis is performed on the bound target sequences. Data analysis is performed, for example, to identify a SNP or CNV as found in a captured target sequence. Data analysis is performed using any array scanner, for example an Axon GenePix 4000B fluorescent scanner. Once data is captured by the scanner, bioinformatics programs are utilized to analyze the captured data. Bioinformatics programs useful in data analysis from fluorescent microarray formats include, but are not limited to SignalMap™ (NimbleGen) and NimbleScan™ (NimbleGen) however any scanner and bioinformatics programs capable of capturing and analyzing data generated by the methods of the present invention are equally amenable. Data output is visualized on, for example, any computer screen or other device capable of displaying data generated when practicing the present invention.

In some embodiments, the present invention provides kits for practicing methods and assays as described herein. In some embodiments, the kits comprise reagents and/or other components (e.g., buffers, instructions, solid surfaces, containers, software, etc.) sufficient for, necessary for, performing target nucleic acid capture of target nucleic acid molecules as herein described. Kits are provided to a user in one or more containers (further comprising one or more tubes, packages, etc.) that may require differential storage, for example differential storage of kit components/reagents due to light, temperature, etc. requirements particular to each kit component/reagent. In some embodiments, a kit comprises one or more solid supports, wherein said solid support is a microarray slide or a plurality of beads, upon which are affixed a plurality of oligonucleotide capture probes. In some embodiments, a kit comprises oligonucleotide probes in solution, wherein said probes comprise a capture moiety, and beads, wherein said beads are designed to bind to the capture moiety as affixed to the oligonucleotide probe. For example, such a moiety is a biotin label which can be used for immobilization on a streptavidin coated solid support. Alternatively, such a modification is a hapten like digoxygenin, which can be used for immobilization on a solid support coated with a hapten recognizing antibody.

In some embodiments, the kit of the present invention comprises at least one or more compounds and reagents for performing enzymatic reactions, for example one or more of a flap endonuclease, a DNA ligase, a RecJ exonuclease, a ssDNA binding protein, a T4 polynucleotide kinase, a restriction endonuclease, a DNA polymerase, a terminal transferase, Klenow, etc. In some embodiments, a kit comprises one or more of hybridization solutions, wash solutions, and/or elution reagents. Examples of wash solutions found in a kit include, but are not limited to, Wash Buffer I (0.2×SSC, 0.2% (v/v) SDS, 0.1 mM DTT), and/or Wash Buffer II (0.2×SSC, 0.1 mM DTT) and/or Wash Buffer III (0.5×SSC, 0.1 mM DTT). In some embodiments, one or more buffers or solutions as found in the kit comprise lithium. In some embodiments, a kit comprises one or more elution solutions, wherein said elution solutions comprise purified water and/or a solution containing TRIS buffer and/or EDTA, or other low solute solution.

The following examples are provided in order to demonstrate and further illustrate certain embodiments and aspects of the present invention and are not to be construed as limiting the scope thereof.

### EXAMPLE 1

### Target Capture of Creatine Phosphokinase 6 (CPK6) and Restriction Fragmented PCR Amplicons

Experiments were designed to test for ligation efficiencies in methods and assays of the present invention. Experiments were designed utilizing 3′ labeled CPK6 oligonucleotides (Integrated DNA Technologies (IDT), Coralville Iowa) and PCR fragments amplified from E. coli genomic DNA (ATCC No. 700926D-5) yielding amplicons from around 1100 to around 1500 bps (SEQ ID NO: 2, 3 and 4). Oligonucleotide probes of 50 and 60 mers were designed that comprised a 16 bp hairpin. The hairpin sequence used was 5′-CCGGAGGATACTCCGG-3′ (SEQ ID NO: 1), as shown in FIG. 3. Control probes were synthesized that did not contain hairpin structures. A quartet of probes was designed representing all four bases per strand, as such eight total probes were designed (e.g., 4 for each strand of the DNA target) for the target nucleotide within the query sequence. Each quartet per strand contained the same probe sequence except the terminal base on the 3′ end of the 50/60 mer probe. Probes were synthesized in situ at Roche NimbleGen (Madison, Wis.) on an array with a density of 2.1 million probes per array (HD2).

Three PCR fragments were amplified from genomic E. coli DNA using PCR primers;

| | | |
|---|---|---|
| 1277-F | 5′-ATGAGCAACAATGAATTCCA-3′ | (SEQ ID NO: 5) |
| 1277-R | 5′ATGGTCAGCGGATACAGGAA-3′ | (SEQ ID NO: 6) |
| 2205-F | 5′ATGAATGACACCAGCTTCGA-3′ | (SEQ ID NO: 7) |
| 2205-R | 5′GCCAGTAGCGTAATCGGATG-3′ | (SEQ ID NO: 8) |
| 2825-F | 5′-ATGTCTGAACAACACGCACA-3′ | (SEQ ID NO: 9) |
| 2825-R | 5′ACAGAATAACGTCGCGGATG-3′. | (SEQ ID NO: 10) |

PCR amplification conditions included initial denaturation at 94° C. for 2 min followed by 30 cycles of 94° C./30 seconds, 55° C./60 seconds, 72° C. 60, with a final elongation of 72° C. for 7 minutes. The fragments were digested with two restriction enzymes (HhaI and NlaIII) yielding fragments of various sizes with 3′ overhangs. The restriction fragmented amplicons were pooled in equimolar concentrations and further treated with antarctic phosphatase (NEB) to dephosphorylate 5′ end. This prevents self-self ligation and direct non-specific ligation to the probes synthesized on the array. Fragmented and dephosphorylated amplicons were labeled with Cy3-ddCTP on their 3′ ends with terminal transferase (TdT, Roche) and precipitated away from non-labeled fragments using methods known to those skilled in the art, for example as found in Molecular Cloning, A Laboratory Manual, Eds. Sambrook et al., Cold Spring Harbor Press (incorporated herein by reference in its entirety). Labeled fragments were combined with phosphorylated CPK6 oligonucleotides that were labeled on the 3′ end with Cy3 (IDT).

The microarray slides were sealed with NimbleChip™ HX3 mixers (Roche NimbleGen, Madison Wis.). Denatured and labeled target amplicons were applied to a microarray. Probes found on the microarray included those with and without a hairpin. The microarray slides with target sequences were allowed to hybridize overnight at 42° C. in a MAUI™ Hybridization System (BioMicro) under stringent conditions, washed three times with wash buffer and scanned. Ligation was performed using Ampligase® (Epicentre, Madison Wis.) in appropriate buffer and incubation was carried out at 45° C. for 4 hours. The slides were washed three times and the arrays boiled in water with constant stirring for approximately 2 minutes. After boiling, the microarray slides were washed and scanned. Scanning was performed using an Axon GenePix™ 4000B fluorescent scanner. Data captured for each scan and data analysis was performed using SignalMap™ (NimbleGen) and NimbleScan™ (NimbleGen) software applications.

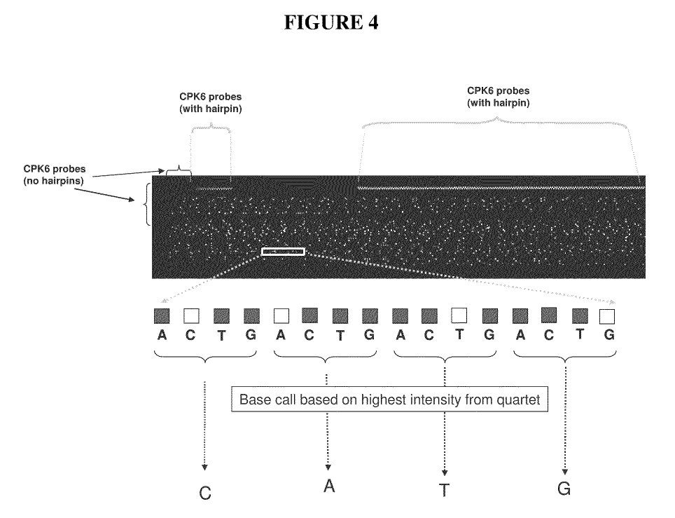

Results demonstrate that for probes with no hairpins (CPK6 control) there was no target capture (FIG. 4). However, when the probe comprised a hairpin and the target fragment sequence comprised the complementary target base, the ligase ligated the target sequence to the probe. The target sequence capture was specific to the identity of the terminal base at the 5′ end of each restriction fragment, such that for each quartet of probes representing a query base for a partial fragment of sequence, only one of the four probes has a ligated labeled fragment. This results in high signal intensity for the correct ligation probe in comparison to the background intensity of the remaining three bases in the quartet per strand. The signal intensities associated with the probe with the correct base perfectly identified the sequence based prediction for locations of restriction sites for HhaI and NlaIII.

### EXAMPLE 2

### Target Capture of Creatine Phosphokinase 6 (CPK6) and PCR Amplicons

Experiments were designed to test for flap endonuclease and ligation efficiencies in methods and assays of the present invention. Experiments were designed utilizing a 3′ labeled CPK6 oligonucleotides (IDT) and PCR fragments amplified from E. coli genomic DNA as described in Example 1. Oligonucleotide probes of 50 and 60 mers were designed that comprised a 16 bp hairpin and a complementary base on the terminal 3′ end. The hairpin sequence used was 5′CCGGAGGATACTCCGG3′ (SEQ ID NO: 1). Control probes were synthesized that did not comprise hairpin structures. A quartet of probes was designed, as exemplified in FIG. 1A, representing all four bases per strand, as such eight total probes were designed (e.g., 4 for each strand of the DNA target) for the target nucleotide within the query sequence. Probes were synthesized in situ at Roche NimbleGen (Madison, Wis.) on an array with a density of 2.1 million probes per array (HD2).

Three PCR fragments were amplified from genomic E. coli DNA using PCR primers and conditions as previously described. The fragments were digested with two restriction enzymes (HhaI and NlaIII) yielding fragments of various sizes with 3′ overhangs. The restriction fragmented amplicons were pooled in equimolar concentrations and further treated with antarctic phosphatase (NEB) to dephosphorylate 5′ end. This prevents self-self ligation and direct non-specific ligation to the probes synthesized on the array. Fragmented and dephosphorylated amplicons were labeled with Cy3-ddCTP on their 3′ ends with terminal transferase (TdT, Roche) and precipitated away from non-labeled fragments using methods known to those skilled in the art, for example as found in Molecular Cloning, A Laboratory Manual, Eds. Sambrook et al., Cold Spring Harbor Press (incorporated herein by reference in its entirety). Labeled fragments were combined with dephosphorylated CPK6 oligonucleotides that were labeled on the 3′ end with Cy3 (IDT).

The microarray slides were sealed with NimbleChip™ HX3 mixers (Roche NimbleGen, Madison Wis.) and denatured, labeled target amplicons were applied to a microarray. Probes found on the microarray included those with and without a hairpin. The microarrays slides with target sequences were allowed to hybridize overnight at 42° C. in a MAUI Hybridization System (BioMicro) under stringent conditions, washed three times with wash buffer and scanned. Cleavase enzyme in appropriate buffer (10 mM MOPs: pH 7.5, 100 mM LiCl, 4 mM MgCl₂ ) was added to the microarray slides with the bound target sequences and the reactions were incubated at 45° C. for 1 hour, washed three times with wash buffer and scanned. Ligation was performed using Ampligase® (Epicentre, Madison Wis.) in appropriate buffer and incubation was carried out at 45° C. for 4 hours. The slides were washed three times and the arrays boiled in water with constant stirring for approximately 2 minutes. After boiling, the microarray slides were washed and scanned. Scanning was performed using an Axon GenePix™ 4000B fluorescent scanner. Data captured for each scan and data analysis was performed using SignalMap™ (NimbleGen) and NimbleScan™ (NimbleGen) software applications.

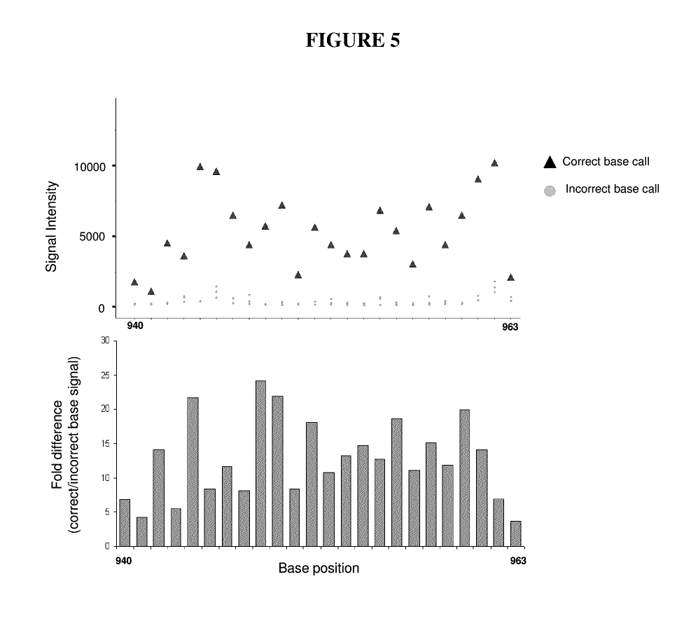

Results demonstrate that for probes with no hairpins (CPK6 control), there was no target capture. However, when the probe comprised a hairpin with a complementary base to the target sequence, and the target sequence comprised the target base, the FEN and ligase enzymes cleaved the target sequence and ligated the target to the probe (respectively). The target sequence capture was specific to the identity of the complement base such that for a quartet of probes representing a query base for a partial fragment of sequence, only one of the four probes had a ligated labeled fragment after cleavase reaction. This results in high signal intensity for the correct base call in comparison to the background intensity of the remaining three bases in the quartet per strand (FIG. 5). The fold differences between correct and incorrect base within the quartet were calculated as the ratio between the signal intensity of the correct base call by the average of three incorrect bases. Fold changes of up to 35 fold were detected across probes that were synthesized on the array to query all fragments of the PCR amplicon, as demonstrated in FIG. 5.

### EXAMPLE 3

### Target Capture of Creatine Phosphokinase 6 (CPK6) and Randomly Fragmented Genomic DNA

Experiments were designed utilizing human and E. coli genomic DNA (gDNA). Oligonucleotide probes were designed as described in Example 1, such that probes were synthesized in situ at Roche NimbleGen (Madison, Wis.) at a density of 2.1 million probes per array (HD2). Genomic DNA (gDNA) was fragmented either with sonication or randomly amplified using Klenow fragment with random primers of various lengths (9 mers, 10 mers, 12 mers, and 15 mers) yielding amplified target sequences of differential lengths. The fragments were treated with antarctic phosphatase and labeled with Cy3-ddCTP on their 3′ ends using terminal transferase (TdT) and precipitated away from non-labeled fragments using methods known to those skilled in the art. The microarray slides were sealed with HX3 mixers (NimbleGen Roche) and denatured, labeled target sequences were applied to a microarray (5-30 μg sample/subarray). Probes found on the microarray included those with and without a complement base immediately after the hairpin on their 3′ ends, as well as probes with no hairpins. The microarray slides with target sequences were allowed to hybridize overnight at 42° C., washed three times with wash buffer and scanned. Cleavase enzyme in appropriate buffer was added to the microarray slides with the bound target sequences and the reactions were incubated at 42° C. for 1-2 hours, washed three times with wash buffer and scanned. Ligation was performed using Ampligase® (Epicentre, Madison Wis.) in appropriate buffer and incubation carried out at 45° C. for 4 hours. The slides were washed three times and the arrays boiled in water with constant stirring for approximately 2 minutes. After boiling, the microarray slides were washed and scanned using an Axon GenePix™ 4000B fluorescent scanner. Data captured by the scanner and data analysis was performed using SignalMap™ (Roche NimbleGen, Inc.) and NimbleScan™ (Roche NimbleGen, Inc.) software applications.

Results demonstrated that for probes with no hairpins (control), there was no target capture. However, when the probe comprised a hairpin with a complementary base, and the target sequence comprised the target base, the FEN and ligase enzymes provided assay specificity in identifying target captured sequences as determined by fluorescence detection methodologies.

### EXAMPLE 4

### Evaluation of Cleavases with Increasing ssDNA Flap Length

Twenty cleavase enzymes were evaluated for efficacy in the cleavase reactions; arbitrarily named C1-C5, P1-P3 and F1-F12. The cleavase enzymes were furnished by Third Wave Technologies (Madison, Wis. 53719). Ampligase® (Epicentre, Madison Wis.). Two microarray slides, each containing 12 identical subarrays were utilized for each experiment to cover all of the 20 cleavases assayed, the probes of which were synthesized by MAS using reverse chemistry (synthesis 5′-3′ where the 5′ end of probe was proximal to the substrate) and each of the 12 subarrays contained approximately 120,0000 probes. The probes were designed to represent three different PCR fragments, sense and antisense strand for each of the three fragments. Each probe was designed with sequence specificity correlating to the target amplicon sequences and a 16 bp hairpin (sequence of 5′-CCGGAGGATACTCCGG-3′ (SEQ ID NO: 1 as seen in FIG. 3) with a 3′ overhang representing an A, C T or G for both the sense and antisense strand (therefore, 8 probes per PCR fragment) for the target nucleotide within the query sequence. Control probes were synthesized that did not comprise hairpin structures.

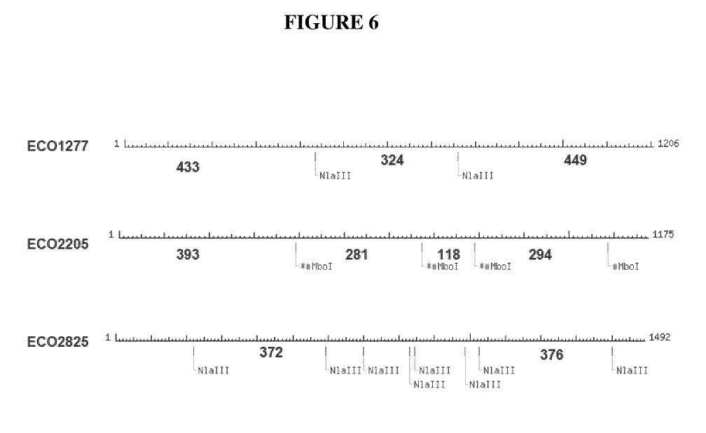

Three different fragments of E. coli were amplified, yielding 1277, 2205 and 2825 bp amplicons. Each amplicon was further fragmented by restriction digest; the 1277 bp and 2825 bp amplicons were digested with NlaIII and the 2205 bp amplicon was digested with MboI yielding digested fragments of various lengths (FIG. 6). The fragments were treated with antarctic phosphatase (NEB) to dephosphorylate 5′ end. This prevents self-self ligation and direct non-specific ligation to the probes synthesized on the array. Fragmented and dephosphorylated amplicons were labeled with Cy3-ddCTP on their 3′ ends with terminal transferase (TdT, Roche) and precipitated away from non-labeled fragments using methods known to those skilled in the art, for example as found in Molecular Cloning, A Laboratory Manual, Eds. Sambrook et al., Cold Spring Harbor Press (incorporated herein by reference in its entirety).

Labeled target nucleic acids were denatured and applied to the subarray on the microarray slides. The microarrays slides with target sequences were allowed to hybridize overnight at 42° C. in a MAUI™ Hybridization System (BioMicro) under stringent conditions, washed three times with wash buffer and scanned. One of the 20 experimental cleavase enzymes and Ampligase® in cleavase buffer (final concentration of 10 mM MOPs: pH 7.4, 100 mM LiCl, 4 mM MgCl₂, 1× NAD) and was added to each of the subarrays on the microarray slides with the bound target sequences and the reactions were incubated at 45° C. for 2 hours, washed three times with wash buffer and scanned. Another round of Ampligase®, this time in its appropriate buffer, was added to each subarray and incubation was carried out at 45° C. for an additional 4 hours. The slides were washed three times and the arrays boiled in water with constant stirring for approximately 2 minutes. After boiling, the microarray slides were again washed and scanned. Scanning was performed using an Axon GenePix™ 4000B fluorescent scanner. Data captured for each scan and data analysis was performed using SignalMap™ (Roche NimbleGen, Inc.) and NimbleScan™ (Roche NimbleGen, Inc.) software applications.

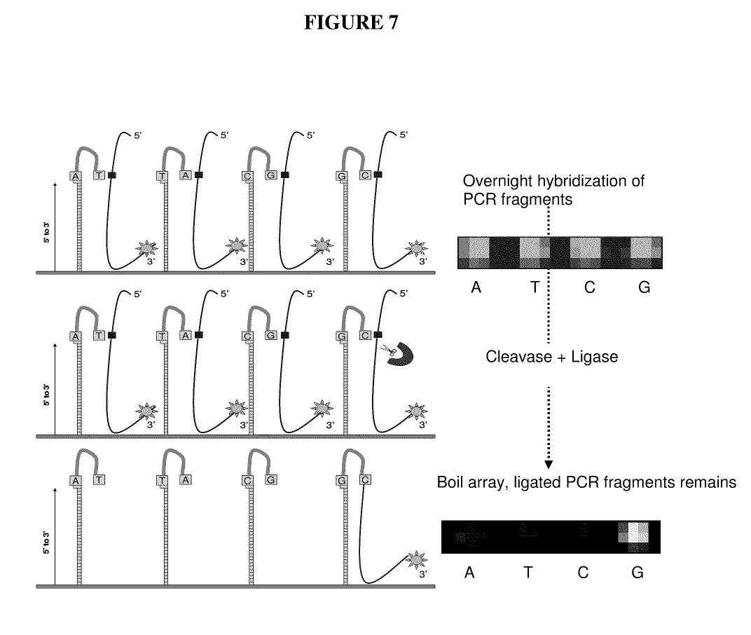

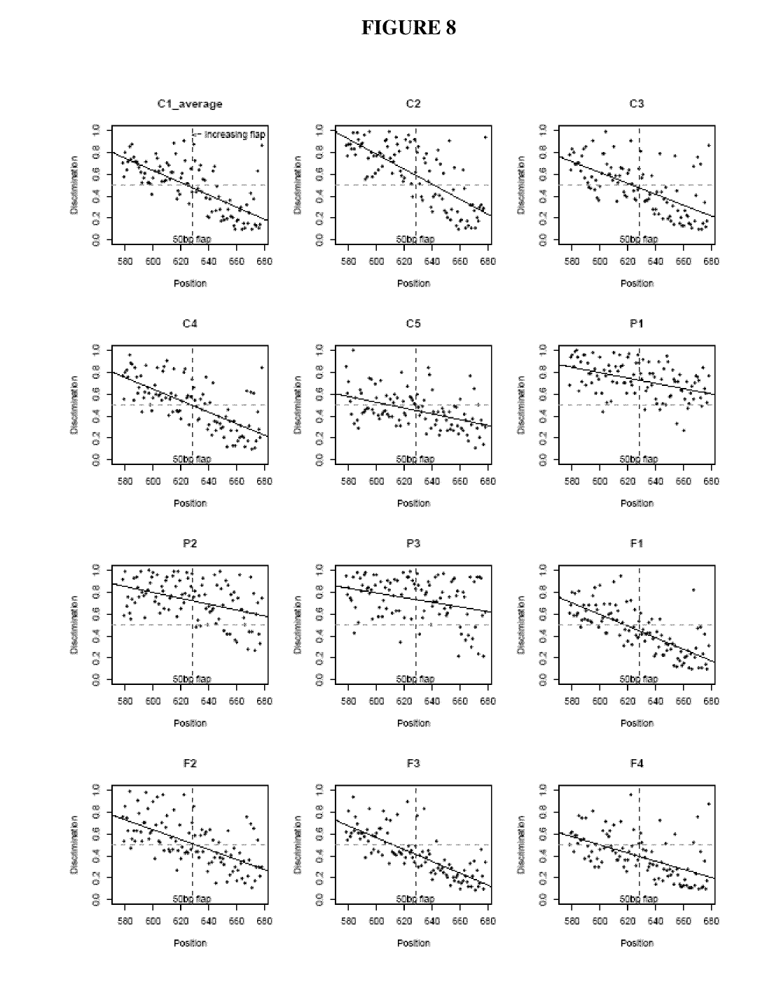

FIG. 7 demonstrates the efficacy of the cleavase/ligase combination in determining genetic sequence of, in this case, a PCR fragment wherein the target sequence had a “C” in the interrogation position. Upon overnight hybridization, there was no discrimination among the bound targets for the probe sequence, whereas incorporation of cleavase and ligase dramatically increased the discrimination resulting in correct base calling at that location. Further, FIG. 8 demonstrates that as the length of the 5′ end of the bound target fragment increases (e.g., flap length increases), an increase in incorrect base calling occurs. Base calling, or discrimination at a single nucleotide, can be determined by the calculation:

$$D = ( \frac{Signal {Intensity}^{SecondBrightestProbe}}{Signal {Intensity}^{BrightestProbe}} )$$

A low discrimination score denotes increased confidence for a correct base call and typically as the flap length increases so does the discrimination score, as such so does the incidence of incorrect base calling. FIG. 8 demonstrates this phenomenon as exemplified with twelve of the difference cleavase molecules evaluated using 50 bp flap length as point of reference and <0.5D score as correct base calling.

### EXAMPLE 5

### Evaluation of the Effect of RecJ on Cleavase Activity

Three PCR fragments as described in Example 4 were utilized to evaluate the ability of RecJ to increase the activity of cleavases for digesting longer 5′ target flap ends. Experimental parameters as found in Example 4 were followed with the following exception. After the overnight hybridization and stringency washes, approximately 120 Units of RecJ_{f} (New England Biolabs, Ipswitch Mass.; a recombinant fusion protein of RecJ and a maltose binding protein (MBP) which retains the same enzymatic properties as wild-type RecJ (MBP added to enhance RecJ solubility)) in RecJ buffer was added to the arrays followed by incubation at 37° C. for 1 hour. Addition of a cleavase and thermostable ligase were as previously described. Arrays were analyzed as previously described.

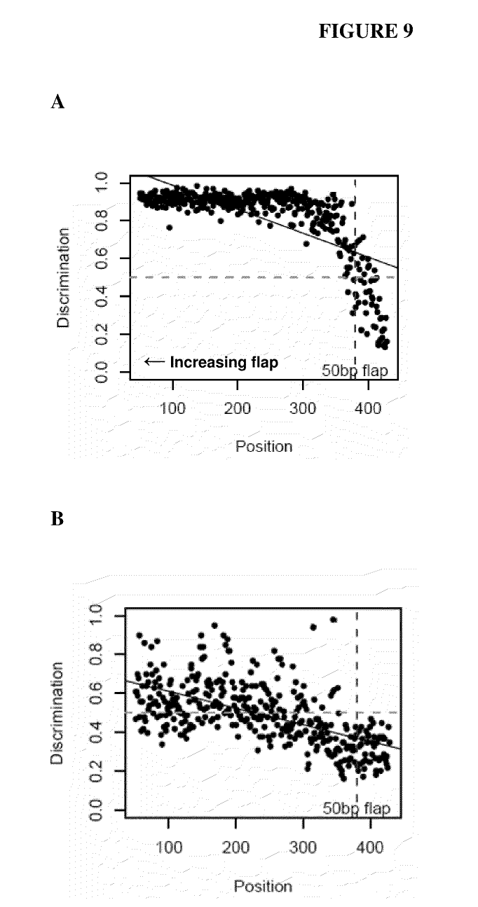

The addition of RecJ in conjunction with a cleavase enzyme greatly increased the activity of the cleavase. As seen in FIG. 9B, treatment of the hybridized complexes with cleavase in conjunction with RecJ increased the activity of the cleavase as compared to treatment with cleavase without RecJ (FIG. 9A). Without RecJ, as the flap length increased, the incidence of incorrect base calling increased such that a flap length of greater than approximately 45 bp led to an increased D score. Conversely, in the presence of RecJ the cleavase activity was maintained due to the activity of RecJ on the target flap overhang, with resultant D scores of around 0.5 or lower resulting in an increase in correct base calls. As such, the inclusion of RecJ in conjunction with a cleavase greatly improves the incidence of correct base calling in a target sample regardless of the length of the target flap overhang.

### EXAMPLE 6

### Positioning of the Interrogation Nucleotide on the 5′-Side of the Probe

Experiments were performed to evaluate the consequences of repositioning the interrogation nucleotide on the probe and its effect on correct base calling for genomic mutation detection. Instead of positioning the interrogation nucleotide after the hairpin structure and at the 3′ end of the probe (as exemplified in FIGS. 1, 2A and 3), the interrogation nucleotide was instead placed immediately prior to the hairpin structure on the 5′ side (or 5′ arm) (FIG. 10) and the 3′ end of the probe was designed to be complementary to the known target sequence. It was contemplated that by positioning the interrogation nucleotide adjacent to the hairpin structure, a dual specificity with respect to both the cleavase and the ligase enzymes is achieved as compared to just the cleavase lending specificity to the detection of mutations, including SNPs. By positioning the interrogation nucleotide as described, the cleavase lends specificity as its activity for detecting and cleaving tripartite structures and the ligase will ligate those cleaved products where the interrogation nucleotide is hybridized to its complement. As such, it is contemplated that a dual specificity is provided resulting in a decrease in false positive base calling of a sample.

FIG. 10 illustrates a comparison of hairpin configuration for single vs. dual enzymatic specificity in cleavase and ligase reactions. Hairpin configuration has an oligomer probe synthesized from 5′-3′ with a hairpin of stem-length (e.g., 6 bp-12 bp) with a 1 bp overhang on the 3′ end (FIG. 10A, C). For each query base, 4 probes are synthesized with hairpins with a single base pair overhang, each representing A, C, T and G. The 3′ end of the probe sequence also complements the overhang on the 3′ end of the hairpin. Hairpin configuration in FIG. 10B, D has an oligomer probe synthesized from 5′-3′ with a hairpin of stem-length (e.g., 6 bp-12 bp). For each query base, 4 probes are synthesized where the last base pair of the hairpin sequence is designed such that it is complementary to the (query base +1) of a known target sequence. For each query base, all four probes have an identical hairpin sequence, but differ in the terminal 3′ end base of the oligomer probes and are synthesized to represent A, C, T and G before synthesis of the hairpin. These generate base-specific substrates for ligation between the 3′ end of the hairpin and the 5′ end of the cleaved target. In the illustrated embodiments shown in FIG. 10, the interrogation nucleotide is positioned immediately prior to the hairpin structure on the 5′-side or arm of the probe. In some embodiments, the proximal nucleotide is about 2, 3, 4, 5, 6, 7, 8, 9, or 10 bases upstream of the double stranded hairpin structure on the 5′ side (i.e., where the probe is single stranded).

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method for capturing target nucleic acid sequences comprising:
a) providing:
i) a nucleic acid sample wherein said nucleic acid sample may or may not comprise a target sequence,
ii) at least one flap endonuclease and at least one ligase, and
iii) a plurality of oligonucleotide probes, wherein said probes comprise target sequences and a hairpin structure,
b) applying said nucleic acid sample to said oligonucleotide probes under conditions wherein hybridization is allowed to occur between target sequences and probes; and
c) applying at least one flap endonuclease and at least one ligase to the hybridized nucleic acid/probes complex under conditions wherein enzymatic reactions are allowed to occur thereby capturing a nucleic acid target sequence.

2. The method of claim 1, wherein said nucleic acid sample further comprises a detection moiety.

3. The method of claim 2, wherein said detection moiety comprises a fluorescent moiety.

4. The method of claim 3, wherein said fluorescent detection moiety is Cy3.

5. The method of claim 2, wherein said detection moiety is detected using a fluorescent scanner.

6. The method of claim 5, further comprising data analysis of the detected target nucleic acids.

7. The method of claim 1, wherein said nucleic acid sample comprises genomic DNA or a derivative thereof.

8. The method of claim 1, wherein said nucleic acid sample is from a mammal.

9. The method of claim 1, wherein said nucleic acid sample is from a human.

10. The method of claim 1, wherein at least one of said target sequences comprises a single nucleotide polymorphism.

11. The method of claim 1, wherein at least one of said target sequences comprises a genomic copy number variant.

12. The method of claim 1, wherein said hairpin structure comprises SEQ ID NO: 1.

13. The method of claim 1, wherein said ligase is a thermostable ligase.

14. The method of claim 1, wherein said probes are affixed to a substrate.

15. The method of claim 14, wherein said substrate is a microarray slide.

16. The method of claim 1, wherein the plurality of oligonucleotide probes comprises an interrogation nucleotide.

17. The method of claim 16, wherein the interrogation nucleotide is positioned at the terminal 3′ end of the probe.

18. The method of claim 16, wherein the interrogation nucleotide is positioned proximal to the hairpin structure on the 5′-side of the probe.

19. The method of claim 16, wherein the interrogation nucleotide is positioned about 2, 3, 4, 5, 6, 7, 8, 9, or 10 bases upstream of the hairpin structure on the 5′-side of the probe.

20. The method of claim 1 further comprising providing a component selected from the group consisting of RecJ and a single strand binding protein.

21. The method of claim 1, wherein the probes provide dual specificity.

22. A method for capturing target nucleic acids comprising:
a) providing:
i) a nucleic acid sample wherein said nucleic acid sample comprises a detection moiety and may or may not comprise a target sequence and,
ii) at least one flap endonuclease, at least one ligase, and
iii) a plurality of oligonucleotide probes, wherein said probes comprise target sequences and a hairpin structure wherein said hairpin structure comprises at least one cleavable sequence,
b) applying said nucleic acid sample to said oligonucleotide probes under conditions wherein hybridization is allowed to occur between target sequences and probes,
c) applying at least one flap endonuclease and at least one ligase to the hybridized nucleic acid/probes complex under conditions wherein enzymatic reactions are allowed to occur thereby capturing a nucleic acid target sequence.

23. The method of claim 22, wherein said at least one cleavable sequence comprises a restriction endonuclease site.

24. The method of claim 22, further comprising releasing of the nucleic acid target sequences from the probes by digestion with a restriction endonuclease.

25. The method of claim 24, further comprising detecting said released target nucleic acid sequences by sequencing.

26. A composition for sequence specific nucleic acid capture comprising a flap endonuclease, a ligase, and an oligonucleotide probe wherein said probe comprises a hairpin structure and a complementary target nucleic acid sequence.

27. A kit for capturing and detecting nucleic acid sequences comprising:
a) at least one flap endonuclease,
b) at least one thermostable ligase,
c) a plurality of oligonucleotide probes affixed to a substrate, and
d) at least one buffer.

28. The kit of claim 27 further comprising a component selected from the group consisting of RecJ and a single strand binding protein.


SEQUENCE LISTING (42 sequences)

SEQ ID NO: 1 - DNA, 16 bp, Artificial
  Oligonucleotide
ccggaggatactccgg

SEQ ID NO: 2 - DNA, 1492 bp, Escherichia coli
atgtctgaacaacacgcacagggcgctgacgcggtagtcgatcttaacaatgaactgaaaacgcgtcgtgagaagctggcgaacctgcgcgagcaggggattgccttcccgaacgatttccgtcgcgatcatacctctgaccaattgcacgcagaattcgacggcaaagagaacgaagaactggaagcgctgaacatcgaagtcgccgttgctggccgcatgatgacccgtcgtattatgggtaaagcgtctttcgttaccctgcaggacgttggcggtcgcattcagctgtacgttgcccgtgacgatctcccggaaggcgtttataacgagcagttcaaaaaatgggacctcggcgacatcctcggcgcgaaaggtaagctgttcaaaaccaaaaccggcgaactgtctatccactgcaccgagttgcgtctgctgaccaaagcactgcgtccgctgccggataaattccacggcttgcaggatcaggaagcgcgctatcgtcagcgttatctcgatctcatctccaacgatgaatcccgcaacacctttaaagtgcgctcgcagatcctctctggtattcgccagttcatggtgaaccgcggctttatggaagttgaaacgccgatgatgcaggtgatccctggcggtgccgctgcgcgtccgtttatcacccaccataacgcgctggatctcgacatgtacctgcgtatcgcgccggaactgtacctcaagcgtctggtggttggtggcttcgagcgtgtattcgaaatcaaccgtaacttccgtaacgaaggtatttccgtacgtcataacccagagttcaccatgatggaactctacatggcttacgcagattacaaagatctgatcgagctgaccgaatcgctgttccgtactctggcacaggatattctcggtaagacggaagtgacctacggcgacgtgacgctggacttcggtaaaccgttcgaaaaactgaccatgcgtgaagcgatcaagaaatatcgcccggaaaccgacatggcggatctggacaacttcgactctgcgaaagcaattgctgaatctatcggcatccacgttgagaagagctggggtctgggccgtatcgttaccgagatcttcgaagaagtggcagaagcacatctgattcagccgaccttcattactgaatatccggcagaagtttctccgctggcgcgtcgtaacgacgttaacccggaaatcacagaccgctttgagttcttcattggtggtcgtgaaatcggtaacggctttagcgagctgaatgacgcggaagatcaggcgcaacgcttcctggatcaggttgccgcgaaagacgcaggtgacgacgaagcgatgttctacgatgaagattacgtcaccgcactggaacatggcttaccgccgacagcaggtctgggaattggtatcgaccgtatggtaatgctgttcaccaacagccataccatccgcgacgttattctgt

SEQ ID NO: 3 - DNA, 1206 bp, Escherichia coli
gagcaacaatgaattccatcagcgtcgtctttctgccactccgcgcggggttggcgtgatgtgtaacttcttcgcccagtcggctgaaaacgccacgctgaaggatgttgagggcaacgagtacatcgatttcgccgcaggcattgcggtgctgaataccggacatcgccaccctgatctggtcgcggcggtggagcagcaactgcaacagtttacccacaccgcgtatcagattgtgccgtatgaaagctacgtcaccctggcggagaaaatcaacgcccttgccccggtgagcgggcaggccaaaaccgcgttcttcaccaccggtgcggaagcggtggaaaacgcggtgaaaattgctcgcgcccataccggacgccctggcgtgattgcgtttagcggcggctttcacggtcgtacgtatatgaccatggcgctgaccggaaaagttgcgccgtacaaaatcggcttcggcccgttccctggttcggtgtatcacgtaccttatccgtcagatttacacggcatttcaacacaggactccctcgacgccatcgaacgcttgtttaaatcagacatcgaagcgaagcaggtggcggcgattattttcgaaccggtgcagggcgagggcggtttcaacgttgcgccaaaagagctggttgccgctattcgccgcctgtgcgacgagcacggtattgtgatgattgctgatgaagtgcaaagcggctttgcgcgtaccggtaagctgtttgccatggatcattacgccgataagccggatttaatgacgatggcgaaaagcctcgcgggcgggatgccgctttcgggcgtggtcggtaacgcgaatattatggacgcacccgcgccgggcgggcttggcggcacctacgccggtaacccgctggcggtggctgccgcgcacgcggtgctcaacattatcgacaaagaatcactctgcgaacgcgcgaatcaactgggccagcgtctcaaaaacacgttgattgatgccaaagaaagcgttccggccattgctgcggtacgcggcctggggtcgatgattgcggtagagtttaacgatccgcaaacgggcgagccgtcagcggcgattgcacagaaaatccagcaacgcgcgctggcgcaggggctgctcctgctgacctgtggcgcatacggcaacgtgattcgcttcctgtatccgctgaccat

SEQ ID NO: 4 - DNA, 1175 bp, Escherichia coli
atgaatgacaccagcttcgaaaactgcattaagtgcaccgtctgcaccaccgcctgcccggtgagccgggtgaatcccggttatccagggccaaaacaagccgggccggatggcgagcgtctgcgtttgaaagatggcgcactgtatgacgaggcgctgaaatattgcatcaactgcaaacgttgtgaagtcgcctgcccgtccgatgtgaagattggcgatattatccagcgcgcgcgggcgaaatatgacaccacgcgcccgtcgctgcgtaattttgtgttgagtcataccgacctgatgggtagcgtttccacgccgttcgcaccaatcgtcaacaccgctacctcgctgaaaccggtgcggcagctgcttgatgcggcgttaaaaatcgatcatcgccgcacgctaccgaaatactccttcggcacgttccgtcgctggtatcgcagcgtggcggctcagcaagcacaatataaagaccaggtcgctttctttcacggctgcttcgttaactacaaccatccgcagttaggtaaagatttaattaaagtgctcaacgcaatgggtaccggtgtacaactgctcagcaaagaaaaatgctgcggcgtaccgctaatcgccaacggctttaccgataaagcacgcaaacaggcaattacgaatgtagagtcgatccgcgaagctgtgggagtaaaaggcattccggtgattgccacctcctcaacctgtacatttgccctgcgcgacgaatacccggaagtgctgaatgtcgacaacaaaggcttgcgcgatcatatcgaactggcaacccgctggctgtggcgcaagctggacgaaggcaaaacgttaccgctgaaaccgctgccgctgaaagtggtttatcacactccgtgccatatggaaaaaatgggctggacgctctacaccctggagctgttgcgtaacatcccggggcttgagttaacggtgctggattcccagtgctgcggtattgcgggtacttacggtttcaaaaaagagaactaccccacctcacaagccatcggcgcaccactgttccgccagatagaagaaagcggcgcagatctggtggtcaccgactgcgaaacctgtaaatggcagattgagatgtccacaagtcttcgctgcgaacatccgattacgctactggc

SEQ ID NO: 5 - DNA, 20 bp, Artificial
  Oligonucleotide
atgagcaacaatgaattcca

SEQ ID NO: 6 - DNA, 20 bp, Artificial
  Oligonucleotide
atggtcagcggatacaggaa

SEQ ID NO: 7 - DNA, 20 bp, Artificial
  Oligonucleotide
atgaatgacaccagcttcga

SEQ ID NO: 8 - DNA, 20 bp, Artificial
  Oligonucleotide
gccagtagcgtaatcggatg

SEQ ID NO: 9 - DNA, 20 bp, Artificial
  Oligonucleotide
atgtctgaacaacacgcaca

SEQ ID NO: 10 - DNA, 20 bp, Artificial
  Oligonucleotide
acagaataacgtcgcggatg

SEQ ID NO: 11 - DNA, 30 bp, Artificial
  Oligonucleotide
acaggcaattacgaatgccggacgtccggc

SEQ ID NO: 12 - DNA, 30 bp, Artificial
  Oligonucleotide
acaggcaattacgaatcccggacgtccggg

SEQ ID NO: 13 - DNA, 30 bp, Artificial
  Oligonucleotide
acaggcaattacgaataccggacgtccggt

SEQ ID NO: 14 - DNA, 30 bp, Artificial
  Oligonucleotide
acaggcaattacgaattccggacgtccgga

SEQ ID NO: 15 - DNA, 25 bp, Artificial
  Oligonucleotide
tgtccgttaatgcttacatctcagc

SEQ ID NO: 16 - DNA, 29 bp, Artificial
  Oligonucleotide
acaggcaattacgaatgccggacgtccgg

SEQ ID NO: 17 - DNA, 29 bp, Artificial
  Oligonucleotide
acaggcaattacgaatcccggacgtccgg

SEQ ID NO: 18 - DNA, 29 bp, Artificial
  Oligonucleotide
acaggcaattacgaataccggacgtccgg

SEQ ID NO: 19 - DNA, 29 bp, Artificial
  Oligonucleotide
acaggcaattacgaattccggacgtccgg

SEQ ID NO: 20 - DNA, 16 bp, Artificial
  Oligonucleotide
ncggaggatactccgn

SEQ ID NO: 21 - DNA, 18 bp, Artificial
  Oligonucleotide
ngagctcgatagagctcn

SEQ ID NO: 22 - DNA, 20 bp, Artificial
  Oligonucleotide
ngagctgcgatagcagctcn

SEQ ID NO: 23 - DNA, 22 bp, Artificial
  Oligonucleotide
ngagactgcgatagcagtctcn

SEQ ID NO: 24 - DNA, 22 bp, Artificial
  Oligonucleotide
ngagactgcttttgcagtctcn

SEQ ID NO: 25 - DNA, 22 bp, Artificial
  Oligonucleotide
nacgtctgcttttgcagacgtn

SEQ ID NO: 26 - DNA, 17 bp, Artificial
  Oligonucleotide
ccggaggatactccggn

SEQ ID NO: 27 - DNA, 17 bp, Artificial
  Oligonucleotide
gagctcgatagagctcn

SEQ ID NO: 28 - DNA, 19 bp, Artificial
  Oligonucleotide
gagctgcgatagcagctcn

SEQ ID NO: 29 - DNA, 21 bp, Artificial
  Oligonucleotide
gagactgcgatagcagtctcn

SEQ ID NO: 30 - DNA, 21 bp, Artificial
  Oligonucleotide
gagactgcttttgcagtctcn

SEQ ID NO: 31 - DNA, 21 bp, Artificial
  Oligonucleotide
acgtctgcttttgcagacgtn

SEQ ID NO: 32 - DNA, 39 bp, Artificial
  Oligonucleotide
tttttcggttcatgcatgtctctgcatttttgcagagac

SEQ ID NO: 33 - DNA, 42 bp, Artificial
  Oligonucleotide
ctgaataccttgtccatgcatgaaccgtagcgtctgaaataa

SEQ ID NO: 34 - DNA, 34 bp, Artificial
  Oligonucleotide
acaggcaattacgaatgcgagctacgtagctcgc

SEQ ID NO: 35 - DNA, 34 bp, Artificial
  Oligonucleotide
acaggcaattacgaatccgagctacgtagctcgg

SEQ ID NO: 36 - DNA, 34 bp, Artificial
  Oligonucleotide
acaggcaattacgaatacgagctacgtagctcgt

SEQ ID NO: 37 - DNA, 34 bp, Artificial
  Oligonucleotide
acaggcaattacgaattcgagctacgtagctcga

SEQ ID NO: 38 - DNA, 33 bp, Artificial
  Oligonucleotide
acaggcaattacgaatgtgagctacgtagctca

SEQ ID NO: 39 - DNA, 33 bp, Artificial
  Oligonucleotide
acaggcaattacgaatctgagctacgtagctca

SEQ ID NO: 40 - DNA, 33 bp, Artificial
  Oligonucleotide
acaggcaattacgaatatgagctacgtagctca

SEQ ID NO: 41 - DNA, 33 bp, Artificial
  Oligonucleotide
acaggcaattacgaatttgagctacgtagctca

SEQ ID NO: 42 - DNA, 25 bp, Artificial
  Oligonucleotide
tgtccgttaatgcttacgtctcagc